(19) United States
(12) Patent Application Publication
(10) Pub. No.: US 2010/0299773 A1
(43) Pub. Date: Nov. 25, 2010

(54) METHODS AND COMPOSITIONS FOR SELECTING AN IMPROVED PLANT

(75) Inventors: Jennifer R. Hicks, Ames, IA (US); Brenda A. Lowe, Mystic, CT (US); Brian J. Martinell, Mount Horeb, WI (US); John Phillips, Lebanon, IL (US); John P. Tamulonis, Nevada, IA (US); Edward J. Williams, Madison, WI (US)

Correspondence Address: NELSON MULLINS RILEY & SCARBOROUGH, LLP;IP Department, 100 North Tyron Street, 42nd Floor, Charlotte, NC, 28202, US

(73) Assignee: MONSANTO TECHNOLOGY LLC, St. Louis, MO (US)

(21) Appl. No.: 12/778,525

(22) Filed: May 12, 2010

Related U.S. Application Data
(60) Provisional application No. 61/179,913, filed on May 20, 2009.

(51) Int. Cl. C12N 15/82; A01H 5/00; A01H 1/02

(52) U.S. Cl. 800/260; 800/278; 800/279; 800/281; 800/298

(57) ABSTRACT

The invention is directed, in an embodiment, to a method for producing a transgenic plant comprising: providing a substantially homozygous plant line suitable for transformation; selecting a subline of the plant line having reduced heterogeneity; transforming plant materials from the subline with a transgenic construct that confers a desired trait to at least one transformed plant; recovering at least one transgenic event from the transformation step; and selecting a transgenic event exhibiting a desirable level of the desired trait using plants of the subline as control. The invention is also directed to a method for producing a plant having a desired trait comprising: providing a substantially homozygous plant line; selecting a subline of the plant line having reduced heterogeneity; crossing at least one individual of the selected subline with a donor parent having at least one desired trait to form at least one second individual having the desired trait; and backcrossing the at least one second individual with the at least one individual of the selected subline as a recurrent parent to form at least one progeny plant having the desired trait.

### CROSS-REFERENCE TO RELATED APPLICATIONS

This application claims the benefit under 35 U.S.C. §119(e) of U.S. Provisional Application No. 61/179,913 filed May 20, 2009, the entirety of which is hereby incorporated by reference.

### INCORPORATION OF THE SEQUENCE LISTING

A sequence listing is contained in the file named “pa_{—}54014.txt” which is 74,303 bytes (measured in MS-Windows) and was created on Mar. 2, 2009. This electronic sequence listing is electronically filed herewith and is incorporated herein by reference in its entirety.

### FIELD OF THE INVENTION

The present invention relates generally to the field of plant breeding.

### SUMMARY OF THE INVENTION

In an embodiment, the invention is directed to a method for producing a transgenic plant comprising: providing a substantially homozygous plant line suitable for transformation; selecting a subline of the plant line having reduced heterogeneity; transforming plant materials from the subline with a transgenic construct that confers a desired trait to at least one transformed plant; recovering at least one transgenic event from the transformation step; and selecting a transgenic event exhibiting a desirable level of the desired trait using plants of the subline as control.

The invention is also directed, in an embodiments, to a transgenic plant produced according to the method comprising: providing a substantially homozygous plant line suitable for transformation; selecting a subline of the plant line having reduced heterogeneity; transforming plant materials from the subline with a transgenic construct that confers a desired trait to at least one transformed plant; recovering at least one transgenic event from the transformation step; and selecting a transgenic event exhibiting a desirable level of the desired trait using plants of the subline as control, wherein the transformation efficiency of the subline is at least about 20% greater than the transformation efficiency of the substantially homozygous plant line.

In another embodiment, the invention is directed to a method for producing a plant having a desired trait comprising: providing a substantially homozygous plant line; selecting a subline of the plant line having reduced heterogeneity; crossing at least one individual of the selected subline with a donor parent having at least one desired trait to form at least one second individual having the desired trait; and backcrossing the at least one second individual with the at least one individual of the selected subline as a recurrent parent to form at least one progeny plant having the desired trait.

### BRIEF DESCRIPTION OF NUCLEIC ACID SEQUENCES

SEQ ID NO: 1 is a genomic sequence derived from Glycine max associated with locus 1.

SEQ ID NO: 2 is a genomic sequence derived from Glycine max associated with locus 2.

SEQ ID NO: 3 is an alternate genomic sequence derived from Glycine max associated with locus 2.

SEQ ID NO: 4 is a genomic sequence derived from Glycine max associated with locus 3.

SEQ ID NO: 5 is a genomic sequence derived from Glycine max associated with locus 4.

SEQ ID NO: 6 is an alternate genomic sequence derived from Glycine max associated with locus 4.

SEQ ID NO: 7 is a genomic sequence derived from Glycine max associated with locus 5.

SEQ ID NO: 8 is a genomic sequence derived from Glycine max associated with locus 6.

SEQ ID NO: 9 is an alternate genomic sequence derived from Glycine max associated with locus 6.

SEQ ID NO: 10 is a genomic sequence derived from Glycine max associated with locus 6.

SEQ ID NO: 11 is an alternate genomic sequence derived from Glycine max associated with locus 6.

SEQ ID NO: 12 is an alternate genomic sequence derived from Glycine max associated with locus 6.

SEQ ID NO: 13 is an alternate genomic sequence derived from Glycine max associated with locus 6.

SEQ ID NO: 14 is an alternate genomic sequence derived from Glycine max associated with locus 6.

SEQ ID NO: 15 is an alternate genomic sequence derived from Glycine max associated with locus 6.

SEQ ID NO: 16 is an alternate genomic sequence derived from Glycine max associated with locus 6.

SEQ ID NO: 17 is a genomic sequence derived from Glycine max associated with locus 7.

SEQ ID NO: 18 is an alternate genomic sequence derived from Glycine max associated with locus 7.

SEQ ID NO: 19 is a genomic sequence derived from Glycine max associated with locus 8.

SEQ ID NO: 20 is an alternate genomic sequence derived from Glycine max associated with locus 8.

SEQ ID NO: 21 is a genomic sequence derived from Glycine max associated with locus 9.

SEQ ID NO: 22 is an alternate genomic sequence derived from Glycine max associated with locus 9.

SEQ ID NO: 23 is an alternate genomic sequence derived from Glycine max associated with locus 9.

SEQ ID NO: 24 is a forward PCR primer for the amplification of SEQ ID NO: 1.

SEQ ID NO: 25 is a reverse PCR primer for the amplification of SEQ ID NO: 1.

SEQ ID NO: 26 is a forward PCR primer for the amplification of SEQ ID NO: 2.

SEQ ID NO: 27 is a reverse PCR primer for the amplification of SEQ ID NO: 2.

SEQ ID NO: 28 is a forward PCR primer for the amplification of SEQ ID NO: 3.

SEQ ID NO: 29 is a reverse PCR primer for the amplification of SEQ ID NO: 3.

SEQ ID NO: 30 is a forward PCR primer for the amplification of SEQ ID NO: 4.

SEQ ID NO: 31 is a reverse PCR primer for the amplification of SEQ ID NO: 4.

SEQ ID NO: 32 is a forward PCR primer for the amplification of SEQ ID NO: 5.

SEQ ID NO: 33 is a reverse PCR primer for the amplification of SEQ ID NO: 5.

SEQ ID NO: 34 is a forward PCR primer for the amplification of SEQ ID NO: 6.

SEQ ID NO: 35 is a reverse PCR primer for the amplification of SEQ ID NO: 6.

SEQ ID NO: 36 is a forward PCR primer for the amplification of SEQ ID NO: 7.

SEQ ID NO: 37 is a reverse PCR primer for the amplification of SEQ ID NO: 7.

SEQ ID NO: 38 is a forward PCR primer for the amplification of SEQ ID NO: 8.

SEQ ID NO: 39 is a reverse PCR primer for the amplification of SEQ ID NO: 8.

SEQ ID NO: 40 is a forward PCR primer for the amplification of SEQ ID NO: 9.

SEQ ID NO: 41 is a reverse PCR primer for the amplification of SEQ ID NO: 9.

SEQ ID NO: 42 is a forward PCR primer for the amplification of SEQ ID NO: 10.

SEQ ID NO: 43 is a reverse PCR primer for the amplification of SEQ ID NO: 10.

SEQ ID NO: 44 is a forward PCR primer for the amplification of SEQ ID NO: 11.

SEQ ID NO: 45 is a reverse PCR primer for the amplification of SEQ ID NO: 11.

SEQ ID NO: 46 is a forward PCR primer for the amplification of SEQ ID NO: 12.

SEQ ID NO: 47 is a reverse PCR primer for the amplification of SEQ ID NO: 12.

SEQ ID NO: 48 is a forward PCR primer for the amplification of SEQ ID NO: 13.

SEQ ID NO: 49 is a reverse PCR primer for the amplification of SEQ ID NO: 13.

SEQ ID NO: 50 is a forward PCR primer for the amplification of SEQ ID NO: 14.

SEQ ID NO: 51 is a reverse PCR primer for the amplification of SEQ ID NO: 14.

SEQ ID NO: 52 is a forward PCR primer for the amplification of SEQ ID NO: 15.

SEQ ID NO: 53 is a reverse PCR primer for the amplification of SEQ ID NO: 15.

SEQ ID NO: 54 is a forward PCR primer for the amplification of SEQ ID NO: 16.

SEQ ID NO: 55 is a reverse PCR primer for the amplification of SEQ ID NO: 16.

SEQ ID NO: 56 is a forward PCR primer for the amplification of SEQ ID NO: 17.

SEQ ID NO: 57 is a reverse PCR primer for the amplification of SEQ ID NO: 17.

SEQ ID NO: 58 is a forward PCR primer for the amplification of SEQ ID NO: 18.

SEQ ID NO: 59 is a reverse PCR primer for the amplification of SEQ ID NO: 18.

SEQ ID NO: 60 is a forward PCR primer for the amplification of SEQ ID NO: 19.

SEQ ID NO: 61 is a reverse PCR primer for the amplification of SEQ ID NO: 19.

SEQ ID NO: 62 is a forward PCR primer for the amplification of SEQ ID NO: 20.

SEQ ID NO: 63 is a reverse PCR primer for the amplification of SEQ ID NO: 20.

SEQ ID NO: 64 is a forward PCR primer for the amplification of SEQ ID NO: 21.

SEQ ID NO: 65 is a reverse PCR primer for the amplification of SEQ ID NO: 21.

SEQ ID NO: 66 is a forward PCR primer for the amplification of SEQ ID NO: 22.

SEQ ID NO: 67 is a reverse PCR primer for the amplification of SEQ ID NO: 22.

SEQ ID NO: 68 is a forward PCR primer for the amplification of SEQ ID NO: 23.

SEQ ID NO: 69 is a reverse PCR primer for the amplification of SEQ ID NO: 23.

SEQ ID NO: 70 is a probe for the detection of the SNP of SEQ ID NO: 1.

SEQ ID NO: 71 is an alternate probe for the detection of the SNP of SEQ ID NO: 1.

SEQ ID NO: 72 is a probe for the detection of the SNP of SEQ ID NO: 2.

SEQ ID NO: 73 is an alternate probe for the detection of the SNP of SEQ ID NO: 2.

SEQ ID NO: 74 is a probe for the detection of the SNP of SEQ ID NO: 3.

SEQ ID NO: 75 is an alternate probe for the detection of the SNP of SEQ ID NO: 3.

SEQ ID NO: 76 is a probe for the detection of the SNP of SEQ ID NO: 4.

SEQ ID NO: 77 is an alternate probe for the detection of the SNP of SEQ ID NO: 4.

SEQ ID NO: 78 is a probe for the detection of the SNP of SEQ ID NO: 5.

SEQ ID NO: 79 is an alternate probe for the detection of the SNP of SEQ ID NO: 5.

SEQ ID NO: 80 is a probe for the detection of the SNP of SEQ ID NO: 6.

SEQ ID NO: 81 is an alternate probe for the detection of the SNP of SEQ ID NO: 6.

SEQ ID NO: 82 is a probe for the detection of the SNP of SEQ ID NO: 7.

SEQ ID NO: 83 is an alternate probe for the detection of the SNP of SEQ ID NO: 7.

SEQ ID NO: 84 is a probe for the detection of the SNP of SEQ ID NO: 8.

SEQ ID NO: 85 is an alternate probe for the detection of the SNP of SEQ ID NO: 8.

SEQ ID NO: 86 is a probe for the detection of the SNP of SEQ ID NO: 9.

SEQ ID NO: 87 is an alternate probe for the detection of the SNP of SEQ ID NO: 9.

SEQ ID NO: 88 is a probe for the detection of the SNP of SEQ ID NO: 10.

SEQ ID NO: 89 is an alternate probe for the detection of the SNP of SEQ ID NO: 10.

SEQ ID NO: 90 is a probe for the detection of the SNP of SEQ ID NO: 11.

SEQ ID NO: 91 is an alternate probe for the detection of the SNP of SEQ ID NO: 11.

SEQ ID NO: 92 is a probe for the detection of the SNP of SEQ ID NO: 12.

SEQ ID NO: 93 is an alternate probe for the detection of the SNP of SEQ ID NO: 12.

SEQ ID NO: 94 is a probe for the detection of the SNP of SEQ ID NO: 13.

SEQ ID NO: 95 is an alternate probe for the detection of the SNP of SEQ ID NO: 13.

SEQ ID NO: 96 is a probe for the detection of the SNP of SEQ ID NO: 14.

SEQ ID NO: 97 is an alternate probe for the detection of the SNP of SEQ ID NO: 14.

SEQ ID NO: 98 is a probe for the detection of the SNP of SEQ ID NO: 15.

SEQ ID NO: 99 is an alternate probe for the detection of the SNP of SEQ ID NO: 15.

SEQ ID NO: 100 is a probe for the detection of the SNP of SEQ ID NO: 16.

SEQ ID NO: 101 is an alternate probe for the detection of the SNP of SEQ ID NO: 16.

SEQ ID NO: 102 is a probe for the detection of the SNP of SEQ ID NO: 17.

SEQ ID NO: 103 is an alternate probe for the detection of the SNP of SEQ ID NO: 17.

SEQ ID NO: 104 is a probe for the detection of the SNP of SEQ ID NO: 18.

SEQ ID NO: 105 is an alternate probe for the detection of the SNP of SEQ ID NO: 18.

SEQ ID NO: 106 is a probe for the detection of the SNP of SEQ ID NO: 19.

SEQ ID NO: 107 is an alternate probe for the detection of the SNP of SEQ ID NO: 19.

SEQ ID NO: 108 is a probe for the detection of the SNP of SEQ ID NO: 20.

SEQ ID NO: 109 is an alternate probe for the detection of the SNP of SEQ ID NO: 20.

SEQ ID NO: 110 is a probe for the detection of the SNP of SEQ ID NO: 21.

SEQ ID NO: 111 is an alternate probe for the detection of the SNP of SEQ ID NO: 21.

SEQ ID NO: 112 is a probe for the detection of the SNP of SEQ ID NO: 22.

SEQ ID NO: 113 is an alternate probe for the detection of the SNP of SEQ ID NO: 22.

SEQ ID NO: 114 is a probe for the detection of the SNP of SEQ ID NO: 23.

SEQ ID NO: 115 is an alternate probe for the detection of the SNP of SEQ ID NO: 23.

SEQ ID NO: 116 is a genomic sequence derived from Zea mays associated with locus 10.

SEQ ID NO: 117 is an alternate genomic sequence derived from Zea mays associated with locus 10.

SEQ ID NO: 118 is an alternate genomic sequence derived from Zea mays associated with locus 10.

SEQ ID NO: 119 is an alternate genomic sequence derived from Zea mays associated with locus 10.

SEQ ID NO: 120 is a genomic sequence derived from Zea mays associated with locus 11.

SEQ ID NO: 121 is a genomic sequence derived from Zea mays associated with locus 12.

SEQ ID NO: 122 is an alternate genomic sequence derived from Zea mays associated with locus 12.

SEQ ID NO: 123 is an alternate genomic sequence derived from Zea mays associated with locus 12.

SEQ ID NO: 124 is a genomic sequence derived from Zea mays associated with locus 13.

SEQ ID NO:125 is a forward PCR primer for the amplification of SEQ ID NO:116.

SEQ ID NO:126 is a reverse PCR primer for the amplification of SEQ ID NO:116.

SEQ ID NO:127 is a forward PCR primer for the amplification of SEQ ID NO:117.

SEQ ID NO:128 is a reverse PCR primer for the amplification of SEQ ID NO:117.

SEQ ID NO:129 is a forward PCR primer for the amplification of SEQ ID NO:118.

SEQ ID NO:130 is a reverse PCR primer for the amplification of SEQ ID NO:118.

SEQ ID NO:131 is a forward PCR primer for the amplification of SEQ ID NO:119.

SEQ ID NO:132 is a reverse PCR primer for the amplification of SEQ ID NO:119.

SEQ ID NO:133 is a forward PCR primer for the amplification of SEQ ID NO:120.

SEQ ID NO:134 is a reverse PCR primer for the amplification of SEQ ID NO:120.

SEQ ID NO:135 is a forward PCR primer for the amplification of SEQ ID NO:121.

SEQ ID NO:136 is a reverse PCR primer for the amplification of SEQ ID NO:121.

SEQ ID NO:137 is a forward PCR primer for the amplification of SEQ ID NO:122.

SEQ ID NO:138 is a reverse PCR primer for the amplification of SEQ ID NO:122.

SEQ ID NO:139 is a forward PCR primer for the amplification of SEQ ID NO:123.

SEQ ID NO:140 is a reverse PCR primer for the amplification of SEQ ID NO:123.

SEQ ID NO:141 is a forward PCR primer for the amplification of SEQ ID NO:124.

SEQ ID NO:142 is a reverse PCR primer for the amplification of SEQ ID NO:124.

SEQ ID NO:143 is a probe for the detection of the SNP of SEQ ID NO:116.

SEQ ID NO:144 is an alternate probe for the detection of the SNP of SEQ ID NO:116.

SEQ ID NO:145 is a probe for the detection of the SNP of SEQ ID NO:117.

SEQ ID NO:146 is an alternate probe for the detection of the SNP of SEQ ID NO:117.

SEQ ID NO:147 is a probe for the detection of the SNP of SEQ ID NO:118.

SEQ ID NO:148 is an alternate probe for the detection of the SNP of SEQ ID NO:118.

SEQ ID NO:149 is a probe for the detection of the SNP of SEQ ID NO:119.

SEQ ID NO:150 is an alternate probe for the detection of the SNP of SEQ ID NO:119.

SEQ ID NO:151 is a probe for the detection of the SNP of SEQ ID NO:120.

SEQ ID NO:152 is an alternate probe for the detection of the SNP of SEQ ID NO:120.

SEQ ID NO:153 is a probe for the detection of the SNP of SEQ ID NO:121.

SEQ ID NO:154 is an alternate probe for the detection of the SNP of SEQ ID NO:121.

SEQ ID NO:155 is a probe for the detection of the SNP of SEQ ID NO:122.

SEQ ID NO:156 is an alternate probe for the detection of the SNP of SEQ ID NO:122.

SEQ ID NO:157 is a probe for the detection of the SNP of SEQ ID NO:123.

SEQ ID NO:158 is an alternate probe for the detection of the SNP of SEQ ID NO:123.

SEQ ID NO:159 is a probe for the detection of the SNP of SEQ ID NO:124.

SEQ ID NO: 160 is an alternate probe for the detection of the SNP of SEQ ID NO: 124.

### BRIEF DESCRIPTION OF THE DRAWINGS

FIG. 1 is a bar chart which illustrates the average yield (Bu/A) of 48 F_{6:8}-derived sublines of MV0040 across eight locations.

### DETAILED DESCRIPTION OF THE INVENTION

Reference now will be made in detail to the embodiments of the invention, one or more examples of which are set forth below. Each example is provided by way of explanation of the invention, not a limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment.

Thus, it is intended that the present invention covers such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

The definitions and methods provided define the present invention and guide those of ordinary skill in the art in the practice of the present invention. Unless otherwise noted, terms are to be understood according to conventional usage by those of ordinary skill in the relevant art. Definitions of common terms in molecular biology may also be found in Alberts, et al., Molecular Biology of The Cell, 5^{th} Edition, Garland Science Publishing, Inc.: New York, 2007; Rieger et al., Glossary of Genetics: Classical and Molecular, 5th edition, Springer-Verlag: New York, 1991; King et al., A Dictionary of Genetics, 6th ed, Oxford University Press: New York, 2002; and Lewin, Genes IX, Oxford University Press: New York, 2007. The nomenclature for DNA bases as set forth in 37 CFR §1.822 is used.

An “allele” refers to an alternative sequence of a gene at a particular locus on a chromosome; the length of an allele may be as small as 1 nucleotide base, but may also be larger.

A “locus” is a position on a genomic sequence that is usually found by a point of reference, for example, the position of a DNA sequence that is a gene, or part of a gene or intergenic region. In an embodiment, the loci of this invention comprise one or more polymorphisms in a population; thus, alternative alleles may be present in some individuals.

As used herein, “polymorphism” means the presence of one or more variations of a nucleic acid sequence or nucleic acid feature at one or more loci in a population of one or more individuals. The variation may comprise, but is not limited to, one or more base changes, the insertion of one or more nucleotides, or the deletion of one or more nucleotides. A polymorphism may arise from random processes in nucleic acid replication, through mutagenesis, as a result of mobile genomic elements, from copy number variation and during the process of meiosis, such as unequal crossing over, from genome duplication and/or from chromosome breaks and fusions. The variation may be commonly found or may exist at low frequency within a population, the former having greater utility in general plant breeding and the latter may be associated with rare but important phenotypic variation. Useful polymorphisms may include single nucleotide polymorphisms (SNPs), insertions or deletions in DNA sequence (Indels), simple sequence repeats of DNA sequence (SSRs), a restriction fragment length polymorphism, and/or a tag SNP. A genetic marker, a gene, a DNA-derived sequence, a haplotype, a RNA-derived sequence, a promoter, a 5′ untranslated region of a gene, a 3′ untranslated region of a gene, microRNA, siRNA, a quantitative trail locus (QTL), a satellite marker, a transgene, mRNA, ds mRNA, a transcriptional profile, and/or a methylation pattern may also comprise polymorphisms. In addition, the presence, absence, or variation in copy number of the preceding may comprise polymorphisms.

As used herein, “marker” means a detectable characteristic that can be used to discriminate between organisms. Examples of such characteristics may include genetic markers, protein composition, protein levels, oil composition, oil levels, carbohydrate composition, carbohydrate levels, fatty acid composition, fatty acid levels, amino acid composition, amino acid levels, biopolymers, pharmaceuticals, starch composition, starch levels, fermentable starch, fermentation yield, fermentation efficiency, energy yield, secondary compounds, metabolites, morphological characteristics, and/or agronomic characteristics. As used herein, “genetic marker” means polymorphic nucleic acid sequence or nucleic acid feature. A genetic marker may be represented by one or more particular variant sequences, or by a consensus sequence. In another embodiment, a “genetic marker” may be an isolated variant or consensus of such a sequence.

As used herein, “marker assay” means a method for detecting a polymorphism at a particular locus using a particular method, for example, measurement of at least one phenotype (such as seed color, flower color, or other visually detectable trait), restriction fragment length polymorphism (RFLP), single base extension, electrophoresis, sequence alignment, allelic specific oligonucleotide hybridization (ASO), random amplified polymorphic DNA (RAPD), microarray-based technologies, and/or nucleic acid sequencing technologies.

As used herein, “typing” refers to any method whereby the specific allelic form of a given genomic polymorphism is determined. For example, a single nucleotide polymorphism (SNP) is typed by determining which nucleotide is present (adenine (A), thymine (T), cytosine (C), or guanine (G)). If insertion/deletions (Indels) are to be present, they can be typed by a variety of assays including, but not limited to, marker assays.

As used herein, the phrase “adjacent”, when used to describe a nucleic acid molecule that hybridizes to DNA containing a polymorphism, refers to a nucleic acid that hybridizes to DNA sequences that directly abut the polymorphic nucleotide base position. For example, a nucleic acid molecule that can be used in a single base extension assay is “adjacent” to the polymorphism.

As used herein, “interrogation position” refers to a physical position on a solid support that can be queried to obtain genotyping data for one or more predetermined genomic polymorphisms.

As used herein, “consensus sequence” refers to a constructed DNA sequence which identifies SNP and Indel polymorphisms in alleles at a locus. A consensus sequence may be based on either strand of DNA at the locus, and states the nucleotide base of either one of each SNP in the locus and the nucleotide bases of all Indels in the locus. Thus, although a consensus sequence may not be a copy of an actual DNA sequence, a consensus sequence is useful for precisely designing primers and probes for actual polymorphisms in the locus.

As used herein, the term “single nucleotide polymorphism,” also referred to by the abbreviation “SNP,” means a polymorphism at a single site wherein the polymorphism constitutes a single base pair change, an insertion of one or more base pairs, or a deletion of one or more base pairs.

As used herein, the term “haplotype” means a chromosomal region within a haplotype window defined by at least one polymorphic molecular marker. The unique marker fingerprint combinations in each haplotype window define individual haplotypes for that window. Further, changes in a haplotype, brought about by recombination for example, may result in the modification of a haplotype so that it comprises only a portion of the original (parental) haplotype operably linked to the trait, for example, via physical linkage to a gene, QTL, or transgene. Any such change in a haplotype would be included in our definition of what constitutes a haplotype so long as the functional integrity of that genomic region is unchanged or improved.

As used herein, the term “haplotype window” means a chromosomal region that is established by statistical analyses known to those of skill in the art and is in linkage disequilibrium. Thus, identity by state between two inbred individuals (or two gametes) at one or more molecular marker loci located within this region is taken as evidence of identity-by-descent of the entire region. Each haplotype window includes at least one polymorphic molecular marker. Haplotype windows can be mapped along each chromosome in the genome. Haplotype windows are not fixed per se and, given the ever-increasing density of molecular markers, an embodiment of the invention anticipates the number and size of haplotype windows to evolve, with the number of windows increasing and their respective sizes decreasing, thus resulting in an ever-increasing degree confidence in ascertaining identity by descent based on the identity by state at the marker loci.

As used herein, “genotype” means the combination of alleles located on homologous chromosomes that determines a specific characteristic or trait, and it can be indirectly characterized using markers or directly characterized by nucleic acid sequencing. Suitable markers include a genetic marker, or some other type of marker. A genotype may constitute an allele for at least one genetic marker locus or a haplotype for at least one haplotype window. In some embodiments, a genotype may represent a single locus and in others it may represent a genome-wide set of loci. In another embodiment, the genotype can reflect the sequence of a portion of a chromosome, an entire chromosome, a portion of the genome, and the entire genome.

As used herein, “phenotype” means the detectable characteristics of a cell or organism which can be influenced by genotype.

As used herein, “linkage” refers to relative frequency at which types of gametes are produced in a cross. For example, if locus A has genes “A” or “a” and locus B has genes “B” or “b” and a cross between parent 1 with AABB and parent B with aabb will produce four possible gametes where the genes are segregated into AB, Ab, aB and ab. The null expectation is that there will be independent equal segregation into each of the four possible genotypes, for example, with no linkage one quarter of the gametes will be of each genotype. Segregation of gametes into a genotypes differing from one quarter are attributed to linkage.

As used herein, “linkage disequilibrium” is defined in the context of the relative frequency of gamete types in a population of many individuals in a single generation. If the frequency of allele A is p, a is p′, B is q and b is q′, then the expected frequency (with no linkage disequilibrium) of genotype AB is pq, Ab is pq′, aB is p′q and ab is p′q′. Any deviation from the expected frequency is called linkage disequilibrium. Two loci are to be “genetically linked” when they are in linkage disequilibrium.

As used herein, “quantitative trait locus (QTL)” means a locus that controls to some degree numerically representable traits that are usually continuously distributed.

As used herein, the term “crop” or “crop plant” or “plant” means any plant line that has resulted from breeding and selection for superior agronomic performance.

As used herein, the term “soybean” means Glycine max and includes all plant varieties that can be bred with soybean, including wild soybean species.

As used herein, the term “comprising” means “including but not limited to”.

As used herein, the term “elite line” means any line that has resulted from breeding and selection for superior agronomic performance. Non-limiting examples of elite soybean varieties that are commercially available to farmers or soybean breeders include AG00802, A0868, AG0902, A1923, AG2403, A2824, A3704, A4324, A5404, AG5903 and AG6202 (Asgrow Seeds, Des Moines, Iowa, USA); BPR0144RR, BPR 4077NRR and BPR 4390NRR (Bio Plant Research, Camp Point, Ill., USA); DKB17-51 and DKB37-51 (DeKalb Genetics, DeKalb, Ill., USA); and DP 4546 RR, and DP 7870 RR (Delta & Pine Land Company, Lubbock, Tex., USA); JG 03R501, JG 32R606C ADD and JG 55R503C (JGL Inc., Greencastle, Ind., USA); NKS13-K2 (NK Division of Syngenta Seeds, Golden Valley, Minn., USA); 90M01, 91M30, 92M33, 93M11, 94M30, 95M30 and 97B52 (Pioneer Hi-Bred International, Johnston, Iowa, USA); SG4771NRR and SG5161NRR/STS (Soygenetics, LLC, Lafayette, Ind., USA); S00-K5, S11-L2, S28-Y2, S43-B1, S53-A1, S76-L9 and S78-G6 (Syngenta Seeds, Henderson Ky., USA). An elite plant is a representative plant from an elite variety.

Heterogeneity may arise when two parents are polymorphic at a given locus and fixation does not occur during line development or line derivation. Reducing the heterogeneity may enhance the ability to evaluate the effect of a gene, allele, transgene, or transgene insertion event. For example, Lark, et al. (Proc. Natl. Acad. Sci. (USA) 92:4656-4660. 1995) demonstrated that yield QTL in soybeans can be affected by interactions of alleles at different loci. Reducing the heterogeneity within the line may allow for easier evaluation of a trait, transgene, or transformation insertion event.

An important source of experimental error when evaluating transgenic events derives from the comparison of the transgenic events of the transformation line with the transformation line control. Typically, the transformation line control is a bulk seed of the line, which may be segregating for a small number of loci. The original event line is initially selected and created from a single plant and is likely segregating for fewer loci.

The method of the present invention reduces these experimental sources of error, which is important when evaluating the utility of transgenic constructs and events. In the present invention, the method for producing a transgenic plant comprises, as a first step, providing a substantially homozygous plant line suitable for transformation. In an embodiment, the substantially homozygous plant line may be an inbred line or may be of a self-pollinated variety.

Further, the method of the invention involves selecting a subline of the plant line having reduced heterogeneity. The subline may be selected based upon any plant breeding selection method known in the art. In an embodiment, the subline is selected based upon a marker-assisted selection method. For example, marker-assisted selection may be performed on the substantially homozygous plant line and/or its self-pollinated progenies to identify individuals from the self-pollinated progenies that exhibit reduced heterogeneity when compared with the substantially homozygous plant line.

More particularly, a first plant and a second plant may be genotyped to identify regions which are heterozygous. The first plant may then be crossed with the second plant in order to form a segregating population. In another embodiment, the subline may be self-pollinated over one or more generations to improve homozygosicity. The segregating population may then be screened with one or more nucleic acid markers associated with the heterozygous regions. Finally, the subline may be selected as one or more plants in the segregating population having a homozygous state selected from the heterozygous regions identified in the genotyping step.

In another step of an embodiment of the invention, plant materials from the subline are transformed with a transgenic construct that confers a desired trait to at least one transformed plant. The transgenic construct may be inserted using any method known in the art. In an embodiment, the method used to transfer the construct to the transformed plant may be electroporation, microprojectile bombardment, Agrobacterium-mediated transformation or direct DNA uptake by protoplasts.

In a further step of an embodiment of the invention, at least one transgenic event is recovered from the transformation step. The transgenic event may be recovered using any method known in the art currently or yet to be discovered, and the transgenic event may be any known in the art or yet to be discovered. In some embodiments, the transgenic event provides the plant with a trait selected from the group consisting of herbicide tolerance, increased yield, insect control, fungal disease resistance, virus resistance, nematode resistance, bacterial disease resistance, abiotic stress tolerance, quality grain, mycoplasma disease resistance, modified oils production, high oil production, high protein production, germination and seedling growth control, enhanced animal and human nutrition, low raffinose levels, environmental stress resistance, increased digestibility, increased industrial enzymes, increased pharmaceutical proteins, increased peptides and small molecules, improved processing traits, improved flavor, nitrogen fixation, hybrid seed production, and/or reduced allergenicity, biopolymers, and biofuels.

In another step of an embodiment of the invention, a transgenic event which exhibits a desirable level of the desired trait is selected, using plants of the subline as the control. That is, the subline is compared to the transformed plant to determine the efficiency of the transformation process. Using the method of the invention may provide a more meaningful comparison, a more uniform background, less noise, and increased transformation efficiency (number of successful tranformants divided by the amount of DNA used). In some embodiments, the transformation efficiency of the subline is at least equivalent to the substantially homozygous plant line. In other embodiments, the transformation efficiency of the subline is greater than that of the substantially homozygous plant line. The term “transformation efficiency”, as used herein, is interchangeable with the terms “transformation performance” and “transformation frequency”.

In an embodiment, the transformation efficiency in the subline may be about 10% to about 300% greater than the transformation efficiency of the substantially homozygous plant line. In another embodiment, the transformation efficiency in the subline may be about 20% to about 200% greater than the transformation efficiency of the substantially homozygous plant line. In a particular embodiment, the transformation efficiency in the subline may be about 30% to about 100% greater than the transformation efficiency of the substantially homozygous plant line. In a certain embodiment, the transformation efficiency in the subline may be about 78% greater than the transformation efficiency of the substantially homozygous plant line.

In still further embodiments, the transformation efficiency in the subline may be at least about 20% greater than the transformation efficiency of the substantially homozygous plant line. In a still further embodiment, the transformation efficiency in the subline may be at least about 50% greater than the transformation efficiency of the substantially homozygous plant line. In a still further embodiment, the transformation efficiency in the subline may be at least about 75% greater than the transformation efficiency of the substantially homozygous plant line.

In a particular embodiment, the transformed plant has a reduced variability between transgenic events as compared to a plant that is transformed directly from the substantially homozygous plant line. In yet another embodiment, the transformed plant has more consistent expression levels of the desired trait as compared to a plant that is transformed directly from the substantially homozygous plant line.

In another embodiment, the invention is directed to a method for producing a plant that has a desired trait. Similar to the method described above, the steps involve providing a substantially homozygous plant line and selecting a subline of the plant line having reduced heterogeneity. The method also involves crossing at least one individual of the selected subline with a donor parent having at least one desired trait to form at least one second individual having the desired trait. In an embodiment, the selected subline does not contain the desired trait prior to the crossing step.

Further, the method involves backcrossing the at least one second individual with the at least one individual of the selected subline as a recurrent parent to form at least one progeny plant having the desired trait. In some embodiments, the backcrossing step is repeated until the progeny plant has a genotype that is substantially identical to that of the selected subline. In other embodiments, the progeny plant may further be self-fertilized.

In some embodiments of the present invention, the plant produced is a soybean plant. The plant may be selected from the group consisting of members of the genus Glycine, more specifically from the group consisting of Glycine arenaria, Glycine argyrea, Glycine canescens, Glycine clandestine, Glycine curvata, Glycine cyrtoloba, Glycine falcate, Glycine latifolia, Glycine latrobeana, Glycine max, Glycine microphylla, Glycine pescadrensis, Glycine pindanica, Glycine rubiginosa, Glycine soja, Glycine sp., Glycine stenophita, Glycine tabacina and Glycine tomentella.

In other embodiments, the plant produced in the present invention may be selected from the group consisting of maize (Zea mays), cotton (Gossypium hirsutum), peanut (Arachis hypogaea), barley (Hordeum vulgare); oats (Avena sativa); orchard grass (Dactylis glomerata); rice (Oryza sativa, including indica and japonica varieties); sorghum (Sorghum bicolo); sugar cane (Saccharum sp); tall fescue (Festuca arundinacea); turfgrass species (e.g. species: Agrostis stolonifera, Poa pratensis, Stenotaphrum secundatum); wheat (Triticum aestivum), and alfalfa (Medicago sativa), members of the genus Brassica, broccoli, cabbage, carrot, cauliflower, Chinese cabbage, cucumber, dry bean, eggplant, fennel, garden beans, gourd, leek, lettuce, melon, okra, onion, pea, pepper, pumpkin, radish, spinach, squash, sweet corn, tomato, watermelon, ornamental plants, and other fruit, vegetable, tuber, oilseed, and root crops, wherein oilseed crops include soybean, canola, oil seed rape, oil palm, sunflower, olive, corn, cottonseed, peanut, flaxseed, safflower, and coconut.

In an embodiment, QTLs associated with yield can be identified using any method known in the art. In an embodiment, the method may comprise single marker analysis. In other embodiments, a similar analysis can be performed to identify QTLs by taking advantage of the residual heterogeneity and phenotypic differences between sublines. QTLs identified from this analysis may include, but are not limited to yield, transformability, disease resistance, insect resistance, protein composition, oil composition, and agronomic performance.

An identified QTL may be introduced into an elite line. The elite plant line may comprise one or more transgenes conferring herbicide tolerance, increased yield, insect control, fungal disease resistance, virus resistance, nematode resistance, bacterial disease resistance, abiotic stress tolerance, quality grain, mycoplasma disease resistance, modified oils production, high oil production, high protein production, germination and seedling growth control, enhanced animal and human nutrition, low raffinose levels, environmental stress resistance, increased digestibility, increased industrial enzymes, increased pharmaceutical proteins, increased peptides and small molecules, improved processing traits, improved flavor, nitrogen fixation, hybrid seed production, and/or reduced allergenicity, biopolymers, and biofuels. In one embodiment, the herbicide tolerance may be selected from the group consisting of glyphosate, dicamba, glufosinate, sulfonylurea, bromoxynil and norflurazon herbicides. As is known in the art, these traits can be provided by methods of plant biotechnology as transgenes in the plants.

In an embodiment, a QTL allele or alleles can be introduced from any plant that contains that allele (donor) to any recipient plant. In one aspect, the recipient plant can contain additional transformation performance and agronomic performance loci. In another embodiment, the recipient plant can contain a transgene. In yet another embodiment, while maintaining the introduced QTL, the genetic contribution of the plant providing the QTL can be reduced by back-crossing or other suitable approaches. In still another embodiment, the nuclear genetic material derived from the donor material in the plant may be less than or about 50% of the total genetic material, less than or about 25% of the total genetic material, less than or about 13% of the total genetic material, less than or about 5% of the total genetic material, 3% of the total genetic material, 2% of the total genetic material or 1% of the total genetic material, but that genetic material contains the transformation performance and agronomic performance locus or loci of interest.

It is further understood that a plant of the present invention, in an embodiment, may exhibit the characteristics of any relative maturity group. In an aspect, the maturity group is selected from the group consisting of maturity group 000, maturity group 00, maturity group 0, maturity group 1, maturity group 2, maturity group 3, maturity group 4, maturity group 5, maturity group 6, maturity group 7, maturity group 8, maturity group 9, and maturity group 10.

An allele of a QTL can, of course, comprise multiple genes or other genetic factors even within a contiguous genomic region or linkage group, such as a haplotype. As used herein, an allele of a disease resistance locus can therefore encompass more than one gene or other genetic factor where each individual gene or genetic component is also capable of exhibiting allelic variation and where each gene or genetic factor is also capable of eliciting a phenotypic effect on the quantitative trait in question. In an aspect of the present invention the allele of a QTL comprises one or more genes or other genetic factors that are also capable of exhibiting allelic variation. The use of the term “an allele of a QTL” is thus not intended to exclude a QTL that comprises more than one gene or other genetic factor. Specifically, an “allele of a QTL” in the Present in the invention can denote a haplotype within a haplotype window wherein a phenotype can be disease resistance. A haplotype window is a contiguous genomic region that can be defined, and tracked, with a set of one or more polymorphic markers wherein the polymorphisms indicate identity by descent. A haplotype within that window can be defined by the unique fingerprint of alleles at each marker. As defined above, an allele is an alternative sequence of a gene at a particular locus on a chromosome. When all the alleles present at a given locus on a chromosome are the same, that plant is homozygous at that locus. If the alleles present at a given locus on a chromosome differ, that plant is heterozygous at that locus. Plants of the present invention may be homozygous or heterozygous at any particular locus or for a particular polymorphic marker.

As part of the invention, plants or parts thereof may be grown in culture and regenerated. In an embodiment, the plant of the present invention is Glycine max. Methods for the regeneration of Glycine max plants from various tissue types and methods for the tissue culture of Glycine max are known in the art (see, for example, Widholm et al., In Vitro Selection and Culture-induced Variation in Soybean, In Soybean: Genetics, Molecular Biology and Biotechnology, Eds. Verma and Shoemaker, CAB International, Wallingford, Oxon, England (1996)). Regeneration techniques for plants such as Glycine max can use as the starting material a variety of tissue or cell types. With Glycine max in particular, regeneration processes have been developed that begin with certain differentiated tissue types such as meristems. Cartha et al., Can. J Bot. 59:1671-1679 (1981), hypocotyl sections, Cameya et al., Plant Science Letters 21: 289-294 (1981), and stem node segments, Saka et al., Plant Science Letters, 19: 193-201 (1980); Cheng et al., Plant Science Letters, 19: 91-99 (1980). Regeneration of whole sexually mature Glycine max plants from somatic embryos generated from explants of immature Glycine max embryos has been reported (Ranch et al., In Vitro Cellular & Developmental Biology 21: 653-658 (1985). Regeneration of mature Glycine max plants from tissue culture by organogenesis and embryogenesis has also been reported (Barwale et al., Planta 167: 473-481 (1986); Wright et al., Plant Cell Reports 5: 150-154 (1986).

In an embodiment, the present invention provides a more isogenic plant selected for by screening for heterozygous loci in the plant, the selection comprising interrogating genomic nucleic acids for the presence of a marker molecule that is genetically linked to a heterozygous allele in the plant and selecting for the homozygous state.

Nucleic acid molecules or fragments thereof are capable of specifically hybridizing to other nucleic acid molecules under certain circumstances. As used herein, two nucleic acid molecules are capable of specifically hybridizing to one another if the two molecules are capable of forming an anti-parallel, double-stranded nucleic acid structure. A nucleic acid molecule is the “complement” of another nucleic acid molecule if they exhibit complete complementarity. As used herein, molecules are exhibit “complete complementarity” when every nucleotide of one of the molecules is complementary to a nucleotide of the other. Two molecules are “minimally complementary” if they can hybridize to one another with sufficient stability to permit them to remain annealed to one another under at least conventional “low-stringency” conditions. Similarly, the molecules are “complementary” if they can hybridize to one another with sufficient stability to permit them to remain annealed to one another under conventional “high-stringency” conditions. Conventional stringency conditions are described by Sambrook et al., In: Molecular Cloning, A Laboratory Manual, 2nd Edition, Cold Spring Harbor Press, Cold Spring Harbor, N.Y. (1989), and by Haymes et al., In: Nucleic Acid Hybridization, A Practical Approach, IRL Press, Washington, D.C. (1985). Departures from complete complementarity are therefore permissible, as long as such departures do not completely preclude the capacity of the molecules to form a double-stranded structure. In order for a nucleic acid molecule to serve as a primer or probe, it need only be sufficiently complementary in sequence to be able to form a stable double-stranded structure under the particular solvent and salt concentrations employed.

As used herein, a substantially homologous sequence is a nucleic acid sequence that will specifically hybridize to the complement of the nucleic acid sequence to which it is being compared under high stringency conditions. The nucleic-acid probes and primers of the present invention can hybridize under stringent conditions to a target DNA sequence. The term “stringent hybridization conditions” is defined as conditions under which a probe or primer hybridizes specifically with a target sequence(s) and not with non-target sequences, as can be determined empirically. The term “stringent conditions” is functionally defined with regard to the hybridization of a nucleic-acid probe to a target nucleic acid (i.e., to a particular nucleic-acid sequence of interest) by the specific hybridization procedure discussed in Sambrook et al., 1989, at 9.52-9.55. See also, Sambrook et al., 1989 at 9.47-9.52, 9.56-9.58; Kanehisa 1984 Nucl. Acids Res. 12:203-213; and Wetmur et al. 1968 J. Mol. Biol. 31:349-370. Appropriate stringency conditions that promote DNA hybridization are known to those skilled in the art and can be found in Current Protocols in Molecular Biology, John Wiley & Sons, N.Y., 1989, 6.3.1-6.3.6. For example, conditions may comprise 6.0× sodium chloride/sodium citrate (SSC) at about 45° C., followed by a wash of 2.0×SSC at 50° C. The salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either of the temperature or the salt concentration may be held constant while the other variable is changed. For example, hybridization using DNA or RNA probes or primers can be performed at 65° C. in 6×SSC, 0.5% SDS, 5× Denhardt's, 100 μg/mL nonspecific DNA (e.g., sonicated salmon sperm DNA) with washing at 0.5×SSC, 0.5% SDS at 65° C., for high stringency.

It is contemplated that lower stringency hybridization conditions such as lower hybridization and/or washing temperatures can be used to identify related sequences having a lower degree of sequence similarity if specificity of binding of the probe or primer to target sequence(s) is preserved. Accordingly, the nucleotide sequences of the present invention can be used for their ability to selectively form duplex molecules with complementary stretches of DNA, RNA, or cDNA fragments.

As set forth above, various nucleic acid markers may be utilized in the present invention for detection of, selection for, and introgression of reduced heterogeneity in plants. The nucleic acid markers may include SNP (single nucleotide polymorphic) markers. Many SNP markers are available and are useful for fingerprinting, detection of, selection for, and introgression of traits. McCarrol (U.S. application Ser. No. 11/504,538) and Wu (PCT Patent App. No. PCT/US2008/006765), herein incorporated by reference in theirentireties, cite many SNP markers that are known to be useful for plant germplasm.

Additional genetic markers can be used to select plants with an allele of a QTL associated with transformation performance and agronomic performance of the present invention. Examples of public marker databases include, for example: Soybase, an Agricultural Research Service, United States Department of Agriculture.

Genetic markers of the present invention include “dominant” or “codominant” markers. “Codominant markers” reveal the presence of two or more alleles (two per diploid individual). “Dominant markers” reveal the presence of only a single allele. The presence of the dominant marker phenotype (e.g., a band of DNA) is an indication that one allele is present in either the homozygous or heterozygous condition. The absence of the dominant marker phenotype (e.g., absence of a DNA band) is evidence that “some other” undefined allele is present, wherein the other allele may be a deletion. In the case of populations where individuals are predominantly homozygous and loci are predominantly dimorphic, dominant and codominant markers can be equally valuable. As populations become more heterozygous and multiallelic, codominant markers may become more informative of the genotype than dominant markers.

In another embodiment, markers, such as single sequence repeat markers (SSR), AFLP markers, RFLP markers, RAPD markers, phenotypic markers, isozyme markers, single nucleotide polymorphisms (SNPs), insertions or deletions (Indels), single feature polymorphisms (SFPs, for example, as described in Borevitz et al. 2003 Gen. Res. 13:513-523), microarray transcription profiles, DNA-derived sequences, and RNA-derived sequences that are genetically linked to or correlated with alleles of a QTL of the present invention can be utilized.

In one embodiment, nucleic acid-based analyses for the presence or absence of the genetic polymorphism can be used for the selection of seeds in a breeding population. A wide variety of genetic markers for the analysis of genetic polymorphisms are available and known to those of skill in the art. The analysis may be used to select for genes, portions of genes, QTL, alleles, or genomic regions (haplotypes) that comprise or are linked to a genetic marker.

Herein, nucleic acid analysis methods are known in the art and include, but are not limited to, polymerase chain reaction (PCR)-based detection methods (for example, TaqMan assays), microarray methods, and nucleic acid sequencing methods. In one embodiment, the detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means.

A method of achieving such amplification employs PCR (Mullis et al. 1986 Cold Spring Harbor Symp. Quant. Biol. 51:263-273; European Patent 50,424; European Patent 84,796; European Patent 258,017; European Patent 237,362; European Patent 201,184; U.S. Pat. No. 4,683,202; U.S. Pat. No. 4,582,788; U.S. Pat. No. 4,683,194), herein incorporated in their entireties, using primer pairs that are capable of hybridizing to the proximal sequences that define a polymorphism in its double-stranded form.

Polymorphisms in DNA sequences can be detected or typed by a variety of effective methods well known in the art including, but not limited to, those disclosed in U.S. Pat. Nos. 5,468,613 and 5,217,863; 5,210,015; 5,876,930; 6,030,787; 6,004,744; 6,013,431; 5,595,890; 5,762,876; 5,945,283; 5,468,613; 6,090,558; 5,800,944; 5,616,464; 7,312,039; 7,238,476; 7,297,485; 7,282,355; 7,270,981; and 7,250,252 all of which are incorporated herein by reference in their entireties. However, the compositions and methods of this invention can be used in conjunction with any polymorphism typing method to type polymorphisms in plant genomic DNA samples. These plant genomic DNA samples used include, but are not limited to, plant genomic DNA isolated directly from a plant, cloned plant genomic DNA, or amplified plant genomic DNA.

For instance, polymorphisms in DNA sequences can be detected by hybridization to allele-specific oligonucleotide (ASO) probes as disclosed in U.S. Pat. Nos. 5,468,613 and 5,217,863. U.S. Pat. No. 5,468,613 discloses allele specific oligonucleotide hybridizations where single or multiple nucleotide variations in nucleic acid sequence can be detected in nucleic acids by a process in which the sequence containing the nucleotide variation is amplified, spotted on a membrane and treated with a labeled sequence-specific oligonucleotide probe.

Target nucleic acid sequence can also be detected by probe ligation methods as disclosed in U.S. Pat. No. 5,800,944, where sequence of interest is amplified and hybridized to probes followed by ligation to detect a labeled part of the probe.

Microarrays can also be used for polymorphism detection, wherein oligonucleotide probe sets are assembled in an overlapping fashion to represent a single sequence such that a difference in the target sequence at one point would result in partial probe hybridization (Borevitz et al., Genome Res. 13:513-523 (2003); Cui et al., Bioinformatics 21:3852-3858 (2005)). On any one microarray, it is expected there will be a plurality of target sequences, which may represent genes and/or noncoding regions wherein each target sequence is represented by a series of overlapping oligonucleotides, rather than by a single probe. This platform provides for high throughput screening a plurality of polymorphisms. A single-feature polymorphism (SFP) is a polymorphism detected by a single probe in an oligonucleotide array, wherein a feature is a probe in the array. Typing of target sequences by microarray-based methods is disclosed in U.S. Pat. Nos. 6,799,122; 6,913,879; and 6,996,476, herein incorporated by reference in their entireties.

Target nucleic acid sequence can also be detected by probe linking methods as disclosed in U.S. Pat. No. 5,616,464, herein incorporated by reference in its entirety, employing at least one pair of probes having sequences homologous to adjacent portions of the target nucleic acid sequence and having side chains which non-covalently bind to form a stem upon base pairing of the probes to the target nucleic acid sequence. At least one of the side chains has a photoactivatable group which can form a covalent cross-link with the other side chain member of the stem.

Other methods for detecting SNPs and Indels include single base extension (SBE) methods. Examples of SBE methods include, but are not limited, to those disclosed in U.S. Pat. Nos. 6,004,744; 6,013,431; 5,595,890; 5,762,876; and 5,945,283, herein incorporated by reference in their entireties. SBE methods are based on extension of a nucleotide primer that is adjacent to a polymorphism to incorporate a detectable nucleotide residue upon extension of the primer. In certain embodiments, the SBE method uses three synthetic oligonucleotides. Two of the oligonucleotides serve as PCR primers and are complementary to sequence of the locus of soybean genomic DNA which flanks a region containing the polymorphism to be assayed. Following amplification of the region of the genome containing the polymorphism, the PCR product is mixed with the third oligonucleotide (called an extension primer) which is designed to hybridize to the amplified DNA adjacent to the polymorphism in the presence of DNA polymerase and two differentially labeled dideoxynucleosidetriphosphates. If the polymorphism is present on the template, one of the labeled dideoxynucleosidetriphosphates can be added to the primer in a single base chain extension. The allele present is then inferred by determining which of the two differential labels was added to the extension primer. Homozygous samples will result in only one of the two labeled bases being incorporated and thus only one of the two labels will be detected. Heterozygous samples have both alleles present, and will thus direct incorporation of both labels (into different molecules of the extension primer) and thus both labels will be detected.

In another method for detecting polymorphisms, SNPs and Indels can be detected by methods disclosed in U.S. Pat. Nos. 5,210,015; 5,876,930; and 6,030,787, herein incorporated by reference in their entireties, in which an oligonucleotide probe having a 5′fluorescent reporter dye and a 3′quencher dye covalently linked to the 5′ and 3′ ends of the probe. When the probe is intact, the proximity of the reporter dye to the quencher dye results in the suppression of the reporter dye fluorescence, e.g. by Forster-type energy transfer. During PCR forward and reverse primers hybridize to a specific sequence of the target DNA flanking a polymorphism while the hybridization probe hybridizes to polymorphism-containing sequence within the amplified PCR product. In the subsequent PCR cycle, DNA polymerase with 5′→3′ exonuclease activity cleaves the probe and separates the reporter dye from the quencher dye resulting in increased fluorescence of the reporter.

For the purpose of QTL mapping, the markers included should be diagnostic of origin in order for inferences to be made about subsequent populations. SNP markers are ideal for mapping because the likelihood that a particular SNP allele is derived from independent origins in the extant populations of a particular species is very low. As such, SNP markers are useful for tracking and assisting introgression of QTLs, particularly in the case of haplotypes.

The genetic linkage of additional marker molecules can be established by a gene mapping model such as, without limitation, the flanking marker model reported by Lander et al. (Lander et al. 1989 Genetics, 121:185-199), and the interval mapping, based on maximum likelihood methods described therein, and implemented in the software package MAPMAKER/QTL (Lincoln and Lander, Mapping Genes Controlling Quantitative Traits Using MAPMAKER/QTL, Whitehead Institute for Biomedical Research, Massachusetts, (1990)). Additional software includes Qgene, Version 2.23 (1996), Department of Plant Breeding and Biometry, 266 Emerson Hall, Cornell University, Ithaca, N.Y.

A maximum likelihood estimate (MLE) for the presence of a marker is calculated, together with an MLE assuming no QTL effect, to avoid false positives. A log₁₀ of an odds ratio (LOD) is then calculated as: LOD=log₁₀ (MLE for the presence of a QTL/MLE given no linked QTL). The LOD score essentially indicates how much more likely the data are to have arisen assuming the presence of a QTL versus in its absence. The LOD threshold value for avoiding a false positive with a given confidence, for example 95%, depends on the number of markers and the length of the genome. Graphs indicating LOD thresholds are set forth in Lander et al. (1989), and are further described by Arús and Moreno-González, Plant Breeding, Hayward, Bosemark, Romagosa (eds.) Chapman & Hall, London, pp. 314-331 (1993).

Additional models can be used. Many modifications and alternative approaches to interval mapping have been reported, including the use of non-parametric methods (Kruglyak et al. 1995 Genetics, 139:1421-1428). Multiple regression methods or models can be also be used, in which the trait is regressed on a large number of markers (Jansen, Biometrics in Plant Breed, van Oijen, Jansen (eds.) Proceedings of the Ninth Meeting of the Eucarpia Section Biometrics in Plant Breeding, The Netherlands, pp. 116-124 (1994); Weber and Wricke, Advances in Plant Breeding, Blackwell, Berlin, 16 (1994)). Procedures combining interval mapping with regression analysis, whereby the phenotype is regressed onto a single putative QTL at a given marker interval, and at the same time onto a number of markers that serve as ‘cofactors,’ have been reported by Jansen et al. (Jansen et al. 1994 Genetics, 136:1447-1455) and Zeng (Zeng 1994 Genetics 136:1457-1468). Generally, the use of cofactors reduces the bias and sampling error of the estimated QTL positions (Utz and Melchinger, Biometrics in Plant Breeding, van Oijen, Jansen (eds.) Proceedings of the Ninth Meeting of the Eucarpia Section Biometrics in Plant Breeding, The Netherlands, pp. 195-204 (1994), thereby improving the precision and efficiency of QTL mapping (Zeng 1994). These models can be extended to multi-environment experiments to analyze genotype-environment interactions (Jansen et al. 1995 Theor. Appl. Genet. 91:33-3).

Selection of appropriate mapping populations is important to map construction. The choice of an appropriate mapping population depends on the type of marker systems employed (Tanksley et al., Molecular mapping in plant chromosomes. chromosome structure and function: Impact of new concepts J. P. Gustafson and R. Appels (eds.). Plenum Press, New York, pp. 157-173 (1988)). Consideration must be given to the source of parents (adapted vs. exotic) used in the mapping population. Chromosome pairing and recombination rates can be severely disturbed (suppressed) in wide crosses (adapted×exotic) and generally yield greatly reduced linkage distances. Wide crosses will usually provide segregating populations with a relatively large array of polymorphisms when compared to progeny in a narrow cross (adapted×adapted).

In an embodiment, an F₂ population is the first generation of selfing. Usually a single F₁ plant is selfed to generate a population segregating for all the genes in Mendelian (1:2:1) fashion. Maximum genetic information is obtained from a completely classified F₂ population using a codominant marker system (Mather, Measurement of Linkage in Heredity: Methuen and Co., (1938)). In the case of dominant markers, progeny tests (e.g. F₃, BCF₂) are required to identify the heterozygotes, thus making it equivalent to a completely classified F₂ population. However, this procedure is often prohibitive because of the cost and time involved in progeny testing. Progeny testing of F₂ individuals is often used in map construction where phenotypes do not consistently reflect genotype (e.g. disease resistance) or where trait expression is controlled by a QTL. Segregation data from progeny test populations (e.g. F₃ or BCF₂) can be used in map construction. Marker-assisted selection can then be applied to cross progeny based on marker-trait map associations (F₂, F₃), where linkage groups have not been completely disassociated by recombination events (i.e., maximum disequilibrium).

Recombinant inbred lines (RIL) (genetically related lines; usually >F₅, developed from continuously selfing F₂ lines towards homozygosity) can be used as a mapping population. Information obtained from dominant markers can be maximized by using RIL because most or all loci are homozygous. Under conditions of tight linkage (i.e., about <10% recombination), dominant and co-dominant markers evaluated in RIL populations provide more information per individual than either marker type in backcross populations (Reiter et al. 1992 Proc. Natl. Acad. Sci. (USA) 89:1477-1481). However, as the distance between markers becomes larger (i.e., loci become more independent), the information in RIL populations decreases dramatically.

Backcross populations (e.g., generated from a cross between a successful variety (recurrent parent) and another variety (donor parent) carrying a trait not present in the former) can be utilized as a mapping population. A series of backcrosses to the recurrent parent can be made to recover most of its desirable traits. Thus, a population is created consisting of individuals nearly like the recurrent parent but each individual carries varying amounts of genomic regions from the donor parent. Backcross populations can be useful for mapping dominant markers if all loci in the recurrent parent are homozygous and the donor and recurrent parent have contrasting polymorphic marker alleles (Reiter et al. 1992). Information obtained from backcross populations using either codominant or dominant markers is less than that obtained from F₂ populations because one, rather than two, recombinant gametes are sampled per plant. Backcross populations, however, are more informative (at low marker saturation) when compared to RILs as the distance between linked loci increases in RIL populations (i.e. about 0.15% recombination). Increased recombination can be beneficial for resolution of tight linkages, but may be undesirable in the construction of maps with low marker saturation.

Near-isogenic lines (NIL) created by many backcrosses to produce an array of individuals that are nearly identical in genetic composition except for the trait or genomic region under interrogation can be used as a mapping population. In mapping with NILs, only a portion of the polymorphic loci are expected to map to a selected region.

Bulk segregant analysis (BSA) is a method developed for the rapid identification of linkage between markers and traits of interest (Michelmore et al. 1991 Proc. Natl. Acad. Sci. (U.S.A.) 88:9828-9832). In BSA, two bulked DNA samples are drawn from a segregating population originating from a single cross. These bulks contain individuals that are identical for a particular trait (resistant or susceptible to particular disease) or genomic region but arbitrary at unlinked regions (i.e. heterozygous). Regions unlinked to the target region will not differ between the bulked samples of many individuals in BSA.

Plants of the present invention can be part of or generated from a breeding program. The choice of breeding method depends on the mode of plant reproduction, the heritability of the trait(s) being improved, and the type of cultivar used commercially (e.g., F₁ hybrid cultivar or pureline cultivar). A cultivar is a race or variety of a plant species that has been created or selected intentionally and maintained through cultivation.

Selected, non-limiting approaches for breeding the plants of the present invention are set forth below. A breeding program can be enhanced using marker-assisted selection (MAS) on the progeny of any cross. It is understood that nucleic acid markers of the present invention can be used in a MAS (breeding) program. It is further understood that any commercial and non-commercial cultivars can be utilized in a breeding program. For example, factors such as emergence vigor, vegetative vigor, abiotic stress tolerance, disease resistance, branching, flowering, seed set, seed size, seed density, standability, and threshability will generally dictate the choice.

For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection. In an aspect, a backcross or recurrent breeding program is undertaken.

The complexity of inheritance influences choice of the breeding method. Backcross breeding can be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes.

Breeding lines can be tested and compared to appropriate standards in environments representative of the commercial target area(s) for two or more generations. The best lines are candidates for new commercial cultivars; those still deficient in traits may be used as parents to produce new populations for further selection.

Pedigree breeding and recurrent selection breeding methods can be used to develop cultivars from breeding populations. Breeding programs combine desirable traits from two or more cultivars or various broad-based sources into breeding pools from which cultivars are developed by selfing and selection of desired phenotypes. New cultivars can be evaluated to determine which have commercial potential.

Backcross breeding has been used to transfer genes for a simply inherited, highly heritable trait into a desirable homozygous cultivar or inbred line, which is the recurrent parent. The source of the trait to be transferred is called the donor parent. After the initial cross, individuals possessing the phenotype of the donor parent are selected and repeatedly crossed (backcrossed) to the recurrent parent. The resulting plant is expected to have most attributes of the recurrent parent (e.g., cultivar) and, in addition, the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

Descriptions of other breeding methods that are commonly used for different traits and crops can be found in one of several reference books (Allard, “Principles of Plant Breeding,” John Wiley & Sons, NY, U. of CA, Davis, Calif., 50-98, 1960; Simmonds, “Principles of crop improvement,” Longman, Inc., NY, 369-399, 1979; Sneep and Hendriksen, “Plant breeding perspectives,” Wageningen (ed), Center for Agricultural Publishing and Documentation, 1979; Fehr, In: Soybeans: Improvement, Production and Uses, 2nd Edition, Manograph., 16:249, 1987; Fehr, “Principles of variety development,” Theory and Technique, (Vol. 1) and Crop Species Soybean (Vol. 2), Iowa State Univ., Macmillan Pub. Co., NY, 360-376, 1987).

Selection of plants for breeding in the present invention is not necessarily dependent on the phenotype of a plant and instead can be based on genetic investigations. For example, one may utilize a suitable genetic marker which is closely genetically linked to a trait of interest. One of these markers may therefore be used to identify the presence or absence of a trait in the offspring of a particular cross, and hence may be used in selection of progeny for continued breeding. This technique may commonly be referred to as marker-assisted selection. Any other type of genetic marker or other assay which is able to identify the relative presence or absence of a trait of interest in a plant may also be useful for breeding purposes. Procedures for marker assisted selection applicable to the breeding of plants are well known in the art. Such methods will be of particular utility in the case of recessive traits and variable phenotypes, or where conventional assays may be more expensive, time consuming or otherwise disadvantageous. Types of genetic markers which could be used in accordance with the invention include, but are not necessarily limited to, Simple Sequence Length Polymorphisms (SSLPs) (Williams et al., 1990), Randomly Amplified Polymorphic DNAs (RAPDs), DNA Amplification Fingerprinting (DAF), Sequence Characterized Amplified Regions (SCARs), Arbitrary Primed Polymerase Chain Reaction (AP-PCR), Amplified Fragment Length Polymorphisms (AFLPs) (EP 534 858, specifically incorporated herein by reference in its entirety), and Single Nucleotide Polymorphisms (SNPs) (Wang et al., 1998).

An alternative to traditional QTL mapping which can be used in the invention involves achieving higher resolution by mapping haplotypes, versus individual markers (Fan et al. 2006 Genetics 172:663-686). This approach tracks blocks of DNA known as haplotypes, as defined by polymorphic markers, which are assumed to be identical by descent in the mapping population. This assumption results in a larger effective sample size, offering greater resolution of QTL. Methods for determining the statistical significance of a correlation between a phenotype and a genotype, in this case a haplotype, may be determined by any statistical test known in the art and with any accepted threshold of statistical significance being required. The application of particular methods and thresholds of significance are well within the skill of the ordinary practitioner of the art.

It is further understood, that the present invention provides bacterial, viral, microbial, insect, mammalian and plant cells comprising the nucleic acid molecules of the present invention.

Many qualitative characters also have potential use as phenotype-based genetic markers in soybeans; however, some or many may not differ among varieties commonly used as parents (Bernard and Weiss, 1973). The most widely used genetic markers are flower color (purple dominant to white), pubescence color (brown dominant to gray), and pod color (brown dominant to tan). The association of purple hypocotyl color with purple flowers and green hypocotyl color with white flowers is commonly used to identify hybrids in the seedling stage. Differences in maturity, height, hilum color, and pest resistance between parents can also be used to verify hybrid plants.

Many useful traits that can be introduced by backcrossing, as well as directly into a plant, are those which are introduced by genetic transformation techniques. Genetic transformation may therefore be used to insert a selected transgene into the soybean variety of the invention or may, alternatively, be used for the preparation of transgenes which can be introduced by backcrossing. Methods for the transformation of many economically important plants, including soybeans, are well known to those of skill in the art. Techniques which may be employed for the genetic transformation of soybeans and other plants include, but are not limited to, electroporation, microprojectile bombardment, Agrobacterium-mediated transformation and direct DNA uptake by protoplasts.

To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wound tissues in a controlled manner.

Protoplasts may also be employed for electroporation transformation of plants (Bates, 1994; Lazzeri, 1995). For example, the generation of transgenic soybean plants by electroporation of cotyledon-derived protoplasts was described by Dhir and Widholm in Intl. Patent Appl. Publ. No. WO 92/17598, the disclosure of which is specifically incorporated herein by reference in its entirety.

A particularly efficient method for delivering transforming DNA segments to plant cells is microprojectile bombardment. In this method, particles are coated with nucleic acids and delivered into cells by a propelling force. Exemplary particles include those comprised of tungsten, platinum, and gold. For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into plant cells by acceleration is the Biolistics Particle Delivery System, which can be used to propel particles coated with DNA or cells through a screen, such as a stainless steel or Nytex screen, onto a surface covered with target cells. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

Microprojectile bombardment techniques are widely applicable, and may be used to transform virtually any plant species. The application of microprojectile bombardment for the transformation of soybeans is described, for example, in U.S. Pat. No. 5,322,783, the disclosure of which is specifically incorporated herein by reference in its entirety.

Agrobacterium-mediated transfer is another widely applicable system for introducing gene loci into plant cells. An advantage of the technique is that DNA can be introduced into whole plant tissues, thereby bypassing the need for regeneration of an intact plant from a protoplast. Modern Agrobacterium transformation vectors are capable of replication in E. coli as well as Agrobacterium, allowing for convenient manipulations (Klee et al., 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described have convenient multi-linker regions flanked by a promoter and a polyadenylation site for direct expression of inserted polypeptide coding genes. Additionally, Agrobacterium containing both armed and disarmed Ti genes can be used for transformation.

In those plant strains where Agrobacterium-mediated transformation is efficient, it is the method of choice because of the facile and defined nature of the gene locus transfer. The use of Agrobacterium-mediated plant integrating vectors to introduce DNA into plant cells is well known in the art (Fraley et al., 1985; U.S. Pat. No. 5,563,055). Use of Agrobacterium in the context of soybean transformation has been described, for example, by Chee and Slightom (1995) and in U.S. Pat. No. 5,569,834 and in U.S. Pat. No. 6,384,301, the disclosures of which are specifically incorporated herein by reference in their entirety.

Transformation of plant protoplasts also can be achieved using methods based on calcium phosphate precipitation, polyethylene glycol treatment, electroporation, and combinations of these treatments (see, e.g., Potrykus et al., 1985; Omirulleh et al., 1993; Fromm et al., 1986; Uchimiya et al., 1986; Marcotte et al., 1988). The demonstrated ability to regenerate soybean plants from protoplasts makes each of these techniques applicable to soybean (Dhir et al., 1991).

Hundreds, if not thousands, of different genes are known and could potentially be introduced into a plant according to the invention. Non-limiting examples of particular genes and corresponding phenotypes one may choose to introduce into a plant are presented below.

### A. Herbicide Resistance

In an embodiment, a gene encoding for herbicide resistance may be introduced into the target plant. Numerous herbicide resistance genes are known and may be employed with the invention. An example is a gene conferring resistance to a herbicide that inhibits the growing point or meristem, such as an imidazalinone or a sulfonylurea. Exemplary genes in this category code for mutant ALS and AHAS enzyme as described, for example, by Lee et al., (1988); Gleen et al., (1992) and Miki et al., (1990), incorporated herein by reference in their entireties.

Resistance genes for glyphosate (resistance conferred by mutant 5-enolpyruvl-3 phosphikimate synthase (EPSP) and aroA genes, respectively) and other phosphono compounds such as glufosinate (phosphinothricin acetyl transferase (PAT) and Streptomyces hygroscopicus phosphinothricin-acetyl transferase (bar) genes) may also be used. See, for example, U.S. Pat. No. 4,940,835 to Shah, et al., incorporated herein by reference in its entirety, which discloses the nucleotide sequence of a form of EPSPS which can confer glyphosate resistance. Examples of specific EPSPS transformation events conferring glyphosate resistance are provided by U.S. Pat. No. 6,040,497, incorporated herein by reference in its entirety.

A DNA molecule encoding a mutant aroA gene can be obtained under ATCC accession number 39256, and the nucleotide sequence of the mutant gene is disclosed in U.S. Pat. No. 4,769,061 to Comai, incorporated herein by reference in its entirety. European patent application No. 0 333 033 to Kumada et al., and U.S. Pat. No. 4,975,374 to Goodman et al., incorporated herein by reference in their entireties, disclose nucleotide sequences of glutamine synthetase genes which confer resistance to herbicides such as L-phosphinothricin. The nucleotide sequence of a phosphinothricin-acetyltransferase gene is provided in European application No. 0 242 246 to Leemans et al. DeGreef et al., (1989), incorporated herein by reference in its entirety, which describes the production of transgenic plants that express chimeric bar genes coding for phosphinothricin acetyl transferase activity. Exemplary of genes conferring resistance to phenoxy propionic acids and cycloshexones, such as sethoxydim and haloxyfop are the Acct-S1, Accl-S2 and Acct-S3 genes described by Marshall et al., (1992), incorporated herein by reference in its entirety.

Genes are also known conferring resistance to a herbicide that inhibits photosynthesis, such as a triazine (psbA and gs+genes) and a benzonitrile (nitrilase gene). Przibila et al., (1991), describe the transformation of Chlamydomonas with plasmids encoding mutant psbA genes. Nucleotide sequences for nitrilase genes are disclosed in U.S. Pat. No. 4,810,648 to Stalker, incorporated herein by reference in its entirety, and DNA molecules containing these genes are available under ATCC Accession Nos. 53435, 67441, and 67442. Cloning and expression of DNA coding for a glutathione S-transferase is described by Hayes et al., (1992), incorporated herein by reference in its entirety.

US Patent Application No: 20030135879, incorporated herein by reference in its entirety, describes isolation of a gene for dicamba monooxygenase (DMO) from Psueodmonas maltophilia which is involved in the conversion of a herbicidal form of the herbicide dicamba to a non-toxic 3,6-dichlorosalicylic acid and thus may be used for producing plants tolerant to this herbicide.

### B. Disease Resistance

In an embodiment, a gene encoding for disease resistance may be introduced into the target plant. Plant defenses are often activated by specific interaction between the product of a disease resistance gene (R) in the plant and the product of a corresponding avirulence (Avr) gene in the pathogen. A plant line of the invention can be transformed with cloned resistance gene to engineer plants that are resistant to specific pathogen strains. See, for example Jones et al., (1994) (cloning of the tomato Cf-9 gene for resistance to Cladosporium fulvum); Martin et al., (1993) (tomato Pto gene for resistance to Pseudomonas syringae pv.); and Mindrinos et al., (1994) (Arabidopsis RSP2 gene for resistance to Pseudomonas syringae).

As part of the invention, a viral-invasive protein or a complex toxin derived there from may also be used for viral disease resistance. For example, the accumulation of viral coat proteins in transformed plant cells imparts resistance to viral infection and/or disease development effected by the virus from which the coat protein gene is derived, as well as by related viruses. See Beachy et al., (1990). Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id.

In an embodiment, a virus-specific antibody may also be used. See, for example, Taviadoraki et al., (1993), incorporated herein by reference in its entirety, which describes how transgenic plants expressing recombinant antibody genes are protected from virus attack.

Similarly, in an embodiment, a barley ribosome-inactivating gene may be utilized to confer increased resistance to fungal disease (Logemann et al., (1992)), incorporated herein by reference in its entirety.

### C. Insect Resistance

In an embodiment, a gene encoding for insect resistance may be introduced into the target plant. One example of an insect resistance gene includes a Bacillus thuringiensis protein, a derivative thereof, or a synthetic polypeptide modeled thereon. See, for example, Geiser et al., (1986), who disclose the cloning and nucleotide sequence of a Bt δ-endotoxin gene. Moreover, DNA molecules encoding δ-endotoxin genes can be purchased from the American Type Culture Collection, Manassas, Va., for example, under ATCC Accession Nos. 40098, 67136, 31995 and 31998. Another example of an insect resistance gene is a lectin. See, for example, Van Damme et al., (1994), who disclose the nucleotide sequences of several Clivia miniata mannose-binding lectin genes. A vitamin-binding protein may also be used, such as avidin. See PCT application US93/06487, the contents of which are hereby incorporated by reference in its entirety. This application teaches the use of avidin and avidin homologues as larvicides against insect pests.

Yet another insect resistance gene is an enzyme inhibitor, such as a protease or proteinase inhibitor or an amylase inhibitor. See, for example, Abe et al., (1987) (nucleotide sequence of rice cysteine proteinase inhibitor), Huub et al., (1993) (nucleotide sequence of cDNA encoding tobacco proteinase inhibitor I), and Sumitani et al., (1993) (nucleotide sequence of Streptomyces nitrosporeus α-amylase inhibitor). An insect-specific hormone or pheromone may also be used. See, for example, the disclosure by Hammock et al., (1990), of baculovirus expression of cloned juvenile hormone esterase, an inactivator of juvenile hormone.

Still other examples include an insect-specific antibody or an immunotoxin derived therefrom and a developmental-arrestive protein. See Taylor et al., (1994), who described enzymatic inactivation in transgenic tobacco via production of single-chain antibody fragments.

### D. Male Sterility

In an embodiment, a gene encoding for male sterility may be introduced into the target plant. Genetic male sterility is available in soybeans and can increase the efficiency with which hybrids are made, in that it can eliminate the need to physically emasculate the soybean plant used as a female in a given cross. (Brim and Stuber, 1973). Herbicide-inducible male sterility systems have also been described. (U.S. Pat. No. 6,762,344).

Where one desires to employ male-sterility systems, it may be beneficial to also utilize one or more male-fertility restorer genes. For example, where cytoplasmic male sterility (CMS) is used, hybrid seed production requires three inbred lines: (1) a cytoplasmically male-sterile line having a CMS cytoplasm; (2) a fertile inbred with normal cytoplasm, which is isogenic with the CMS line for nuclear genes (“maintainer line”); and (3) a distinct, fertile inbred with normal cytoplasm, carrying a fertility restoring gene (“restorer” line). The CMS line is propagated by pollination with the maintainer line, with all of the progeny being male sterile, as the CMS cytoplasm is derived from the female parent. These male sterile plants can then be efficiently employed as the female parent in hybrid crosses with the restorer line, without the need for physical emasculation of the male reproductive parts of the female parent.

The presence of a male-fertility restorer gene results in the production of fully fertile F₁ hybrid progeny. If no restorer gene is present in the male parent, male-sterile hybrids are obtained. Such hybrids are useful where the vegetative tissue of the soybean plant is utilized, but in many cases the seeds will be deemed the most valuable portion of the crop, so fertility of the hybrids in these crops must be restored. Therefore, one aspect of the current invention concerns plants of the soybean variety D5245143 comprising a genetic locus capable of restoring male fertility in an otherwise male-sterile plant. Examples of male-sterility genes and corresponding restorers which could be employed with the plants of the invention are well known to those of skill in the art of plant breeding (see, e.g., U.S. Pat. No. 5,530,191 and U.S. Pat. No. 5,684,242, the disclosures of which are each specifically incorporated herein by reference in their entirety).

### E. Modified Fatty Acid, Phytate and Carbohydrate Metabolism

In an embodiment, a gene encoding for modified fatty acid, phytate, or carbohydrate metabolism may be introduced into the target plant. For example, stearyl-ACP desaturase genes may be used for conferring modified fatty acid metabolism on a plant. See Knutzon et al., (1992). Various fatty acid desaturases have also been described, such as a Saccharomyces cerevisiae OLE1 gene encoding Δ9-fatty acid desaturase, an enzyme which forms the monounsaturated palmitoleic (16:1) and oleic (18:1) fatty acids from palmitoyl (16:0) or stearoyl (18:0) CoA (McDonough et al., 1992); a gene encoding a stearoyl-acyl carrier protein delta-9 desaturase from castor (Fox et al. 1993); Δ6- and Δ12-desaturases from the cyanobacteria Synechocystis responsible for the conversion of linoleic acid (18:2) to gamma-linolenic acid (18:3 gamma) (Reddy et al. 1993); a gene from Arabidopsis thaliana that encodes an omega-3 desaturase (Arondel et al. 1992)); plant Δ9-desaturases (PCT Application Publ. No. WO 91/13972) and soybean and Brassica Δ15 desaturases (European Patent Application Publ. No. EP 0616644).

Phytate metabolism may also be modified by introduction of a phytase-encoding gene to enhance breakdown of phytate, adding more free phosphate to the transformed plant. For example, see Van Hartingsveldt et al., (1993), for a disclosure of the nucleotide sequence of an Aspergillus niger phytase gene. In an embodiment employing soybean plants, this could be accomplished by cloning and then reintroducing DNA associated with the single allele which is responsible for soybean mutants characterized by low levels of phytic acid. See Raboy et al., (2000).

A number of genes are known that may be used to alter carbohydrate metabolism. For example, plants may be transformed with a gene coding for an enzyme that alters the branching pattern of starch. See Shiroza et al., (1988) (nucleotide sequence of Streptococcus mutants fructosyltransferase gene), Steinmetz et al., (1985) (nucleotide sequence of Bacillus subtilis levansucrase gene), Pen et al., (1992) (production of transgenic plants that express Bacillus lichenifonnis α-amylase), Elliot et al., (1993) (nucleotide sequences of tomato invertase genes), Sergaard et al., (1993) (site-directed mutagenesis of barley α-amylase gene), and Fisher et al., (1993) (maize endosperm starch branching enzyme II). The Z10 gene encoding a 10 kD zein storage protein from maize may also be used to alter the quantities of 10 kD Zein in the cells relative to other components (Kirihara et al., 1988).

As used herein, a “nucleic acid molecule,” may be a naturally occurring molecule or “substantially purified”, referring to a molecule separated from substantially all other molecules normally associated with it in its native state. In some embodiments, a substantially purified molecule is the predominant species present in a preparation. A substantially purified molecule may be greater than 60% free, greater than 75% free, greater than 90% free, or greater than 95% free from the other molecules (exclusive of solvent) present in the natural mixture. The term “substantially purified” is not intended to encompass molecules present in their native state.

The agents of the present invention may be “biologically active” with respect to either a structural attribute, such as the capacity of a nucleic acid to hybridize to another nucleic acid molecule, or the ability of a protein to be bound by an antibody (or to compete with another molecule for such binding). Alternatively, such an attribute may be catalytic, and thus involve the capacity of the agent to mediate a chemical reaction or response.

The agents of the present invention may also be recombinant. As used herein, the term recombinant means any agent (e.g. DNA, peptide etc.), that is, or results, however indirect, from human manipulation of a nucleic acid molecule.

The agents of the present invention may be labeled with reagents that facilitate detection of the agent (e.g. fluorescent labels (Prober et al. 1987 Science 238:336-340; Albarella et al., European Patent 144914), chemical labels (Sheldon et al., U.S. Pat. No. 4,582,789; Albarella et al., U.S. Pat. No. 4,563,417), modified bases (Miyoshi et al., European Patent 119448).

Having now generally described the invention, the same will be more readily understood through reference to the following examples which are provided by way of illustration, and are not intended to be limiting of the present invention, unless specified.

### Examples

### Example 1

### Identification of Heterogeneity

In this example, a first soybean plant (MV0110) and a second soybean plant (MV0103) were genotyped to identify regions where they were heterogeneous. The plants were then crossed to form a soybean line MV0040, which produced a good transformation ability and yield potential. The breeding history for MV0040 can be summarized as follows:

| Generation | Season | Year | Bulk/Sps/Prow/Pryt |
|---|---|---|---|
| Cross | Summer | 1996 | |
| F₁ | Fall | 1996 | Bulk |
| F₂ | Winter | 1997 | Bulk |
| F₃ | Summer | 1997 | Bulk |
| F₄ | Summer | 1998 | Progeny Row |
| F₅ | Summer | 1999 | Yield testing |
| F₆ | Summer | 2000 | Yield testing |
| F₇ | Summer | 2001 | Yield testing |
| F₈ | Summer | 2002 | Yield testing |

The parents MV0110 and MV0103, and mother line MV0040 were screened using 1423 SNP markers to determine if the MV0040 contained the heterozygous regions associated with that of the parents. Genetic analysis of the MV0040 line showed that 910 SNP loci were identical to MV0103 and 512 SNP loci were identical to MV0110. Without selection, it would have been expected that 50% of the loci would be attributed to each parent. Twenty-three SNP loci were determined to be heterozygous in MV0040 (Table 1-2).

TABLE 1
Heterozygous Regions in MV0040

| Marker | Region | Locus | Linkage Group | cM | MV0103 | MV0110 | MV0040 |
|---|---|---|---|---|---|---|---|
| NS0099457 | 1 | 1 | A2 | 10.1 | AA | GG | AG |
| NS0097078 | 2 | 2 | A2 | 200 | DD | II | DI |
| NS0102044 | 2 | 3 | A2 | 200.2 | GG | AA | AG |
| NS0100939 | 3 | 4 | B1 | 49.7 | CC | TT | CT |
| NS0119353 | 4 | 5 | B1 | 168.4 | TT | CC | CT |
| NS0116504 | 4 | 6 | B1 | 169 | TT | AA | AT |
| NS0098306 | 5 | 7 | G | 61.5 | AA | CC | AC |
| NS0103073 | 6 | 8 | M | 44.2 | TT | AA | AT |
| NS0113949 | 6 | 9 | M | 44.2 | AA | TT | AT |
| NS0100652 | 6 | 10 | M | 44.2 | AA | TT | AT |
| NS0099639 | 6 | 11 | M | 48.8 | TT | CC | CT |
| NS0124584 | 6 | 12 | M | 56.2 | TT | AA | TT |
| NS0095258 | 6 | 13 | M | 61 | CC | TT | CT |
| NS0124762 | 6 | 14 | M | 64.8 | AA | GG | AG |
| NS0125528 | 6 | 15 | M | 64.8 | CC | GG | CG |
| NS0119106 | 6 | 16 | M | 64.8 | AA | GG | AG |
| NS0116502 | 7 | 17 | D2 | 86.7 | TT | CC | CT |
| NS0119245 | 7 | 18 | D2 | 87.2 | TT | CC | CT |
| NS0120228 | 8 | 19 | D1b + W | 17.5 | GG | AA | AG |
| NS0119813 | 8 | 20 | D1b + W | 21.5 | AA | GG | AG |
| NS0095368 | 9 | 21 | D1b + W | 29.8 | AA | GG | AG |
| NS0093934 | 9 | 22 | D1b + W | 29.9 | AA | GG | AG |
| NS0124203 | 9 | 23 | D1b + W | 32.5 | AA | TT | AT |

TABLE 2
SNP markers for detecting heterozygous loci in MV0040, the allele for each marker
indicated, where “*” designates a one base pair deletion.

| | | | | | | | Forward | Reverse | | | | |
|---|---|---|---|---|---|---|---|---|---|---|---|---|
| | | | Linkage | | SEQ | | Primer | Primer | Probe 1 | | Probe 2 | |
| Marker | Region | Locus | Group | cM | ID | Position | SEQ ID | SEQ ID | SEQ ID | Allele | SEQ ID | Allele |
| NS0099457 | 1 | 1 | A2 | 10.1 | 1 | SNP257 | 24 | 25 | 70 | G | 71 | A |
| NS0097078 | 2 | 2 | A2 | 200 | 2 | IND54 | 26 | 27 | 72 | T | 73 | * |
| NS0102044 | 2 | 3 | A2 | 200.2 | 3 | SNP375 | 28 | 29 | 74 | G | 75 | A |
| NS0100939 | 3 | 4 | B1 | 49.7 | 4 | SNP1346 | 30 | 31 | 76 | T | 77 | C |
| NS0119353 | 4 | 5 | B1 | 168.4 | 5 | SNP599 | 32 | 33 | 78 | T | 79 | C |
| NS0116504 | 4 | 6 | B1 | 169 | 6 | SNP246 | 34 | 35 | 80 | T | 81 | A |
| NS0098306 | 5 | 7 | G | 61.5 | 7 | SNP346 | 36 | 37 | 82 | C | 83 | A |
| NS0103073 | 6 | 8 | M | 44.2 | 8 | SNP96 | 38 | 39 | 84 | T | 85 | A |
| NS0113949 | 6 | 9 | M | 44.2 | 9 | SNP740 | 40 | 41 | 86 | T | 87 | A |
| NS0100652 | 6 | 10 | M | 44.2 | 10 | SNP247 | 42 | 43 | 88 | T | 89 | A |
| NS0099639 | 6 | 11 | M | 48.8 | 11 | SNP362 | 44 | 45 | 90 | T | 91 | C |
| NS0124584 | 6 | 12 | M | 56.2 | 12 | SNP421 | 46 | 47 | 92 | T | 93 | A |
| NS0095258 | 6 | 13 | M | 61 | 13 | SNP80 | 48 | 49 | 94 | T | 95 | C |
| NS0124762 | 6 | 14 | M | 64.8 | 14 | SNP261 | 50 | 51 | 96 | G | 97 | A |
| NS0125528 | 6 | 15 | M | 64.8 | 15 | SNP155 | 52 | 53 | 98 | G | 99 | C |
| NS0119106 | 6 | 16 | M | 64.8 | 16 | SNP417 | 54 | 55 | 100 | G | 101 | A |
| NS0116502 | 7 | 17 | D2 | 86.7 | 17 | SNP408 | 56 | 57 | 102 | T | 103 | C |
| NS0119245 | 7 | 18 | D2 | 87.2 | 18 | SNP1330 | 58 | 59 | 104 | T | 105 | C |
| NS0120228 | 8 | 19 | D1b + W | 17.5 | 19 | SNP91 | 60 | 61 | 106 | G | 107 | A |
| NS0119813 | 8 | 20 | D1b + W | 21.5 | 20 | SNP662 | 62 | 63 | 108 | G | 109 | A |
| NS0095368 | 9 | 21 | D1b + W | 29.8 | 21 | SNP34 | 64 | 65 | 110 | G | 111 | A |
| NS0093934 | 9 | 22 | D1b + W | 29.9 | 22 | SNP51 | 66 | 67 | 112 | T | 113 | A |
| NS0124203 | 9 | 23 | D1b + W | 32.5 | 23 | SNP394 | 68 | 69 | 114 | T | 115 | A |

### Example 2

### Reducing Residual Heterogeneity

As stated in Example 1, 23 SNP loci were determined to be heterozygous in MV0040 (Table 1-2). Two hundred and seventy-five F_{6:7} single plants of MV0040 were fingerprinted using the 23 SNP markers listed in Tables 2. The analysis demonstrated that 55% (±2.9% SE) of the 275 sublines were identical to MV0103 at the 23 loci, 43% (±2.9% SE) of the 275 sublines were identical to MV0110 at the 23 loci, and 2.2% (±2.9% SE) were heterozygous at the 23 loci.

The sublines identified to be heterozygous for a portion of the 23 loci can be self-fertilized and further selected to be homozygous at the 23 loci (this would not be possible for one line). This analysis allows for the selection of sublines that are more isogenic. In summary, the method allows for selection of more isogenic lines that can be used for trait introgression, plant transformation or other similar activities.

Further studies were conducted to confirm reduction in heterogeneity in the sublines compared to the mother line. The mother line (MV0040), parents of the mother line (MV0130 and MV0110) and sublines (MV0112-MV0116) were fingerprinted. The overall heterogeneity was reduced from 4.12% in the mother line MV0040 to 1.15% in MV0112, a 70% reduction in overall heterogeneity (Table 3).

TABLE 3
Analysis of Heterogeneity in mother line and selected sublines

| | % Heterozygous | # Markers |
|---|---|---|
| Line | Markers | Assayed |
| MV0103 | 1.06 | 3156 |
| MV0110 | 4.11 | 3229 |
| MV0040 | 4.12 | 3131 |
| MV0112 | 1.15 | 2864 |
| MV0113 | 1.75 | 1258 |
| MV0114 | 0.89 | 1234 |
| MV0115 | 0.95 | 1258 |
| MV0116 | 0.78 | 1282 |

### Example 3

### Selection of Sublines

Plant breeding develops new, uniform, unique and superior varieties and hybrids. The breeder initially selects and crosses two or more parental lines, followed by repeated self-pollination and selection, producing many new genetic combinations. Each year, the plant breeder selects the germplasm to advance to the next generation. The varieties which are developed are unpredictable. This unpredictability is because the breeder's selection occurs in unique environments, with no control at the DNA level (using conventional breeding procedures), and with millions of different possible genetic combinations being generated. The same breeder cannot produce the same variety twice by using the exact same original parents and the same selection techniques.

One goal of plant breeding is to create a uniform variety. To accomplish this task, breeders commonly harvest one or more pods from each plant in a population and thresh them together to form a bulk. Often pods are bulked from plants which are morphologically similar to produce a uniform variety. A single cross may result in many bulk populations which are morphologically distinct. Selection of soybean plants for breeding is not necessarily dependent on the morphology of a plant and instead can be based on genetic investigations. Sublines of a cross or mother line may be selected by genotyping to identify regions which are heterozygous in the mother line. Next, the mother line is self-pollinated. Seed of the subsequent generation is screened with previously determined markers associated with the heterozygous regions. Populations are selected and bulked based on the heterozygous regions. Individuals may also be bulked with favorable haplotypes which are similar to that of the heterozygous regions for such traits as increased yield or pest resistance. Additionally, individuals may be bulked with similar homozygous regions to generate a more uniform population. In some embodiments, the subline may be used as a progenitor in plant transformation. In other embodiments, the subline may be used as a recurrent parent in backcrossing.

For a given SNP locus, the favorability of the two alleles is determined by testing the lines by SNP allele effect. Alternatively, a method is to select the favorable allele for a given SNP locus by employing an extensive trait-haplotype association database with favorable alleles predetermined by empirical methods (U.S. application Ser. No. 11/204,780, herein incorporated by reference in its entirety).

### Example 4

### Evaluating Soybean Sublines for Heterogeneity for Improving Transformation System

Availability of well-characterized sublines that are isogenic for specific genomic regions may lead to an improved transformation system. Highly homogeneous plant material for transformation may eliminate potential variability between events due to heterogeneous genetic backgrounds. Therefore, selecting a subline with yield potential and transformation performance at least equivalent to the more heterogenic mother lines would provide increased efficiency in the overall transformation system. Furthermore, the residual genetic variation in the mother line may provide improvements over the mother line in traits such as transformation performance.

Sublines of soybean variety MV0040 were evaluated for both residual heterogeneity and differences in traits, such as agronomic performance, resistance, and transformation performance to help identify alleles associated with a trait. Two hundred and seventy-five F_{6:7} single plants of MV0040 were fingerprinted using the 23 SNP markers listed in Tables 1-2. In addition, haplotypes and multiple loci were analyzed in association with transformation performance. Meristem explants from MV0040 haplotype lines and the parent lines MV0103 and MV0110 were transformed with Agrobacterium strain ABI harboring control plasmid. These experiments were designed to give a sufficient number of explants to detect a significant difference in transformation frequency from 1% to 2%. Regenerated shoots were harvested from explants at multiple schedules times to ascertain if any differences in shooting rates between haplotypes could be detected. All R₀ plants were assayed for the backbone sequence from the plasmid. Transformation frequency ranged from 1.73% to 2.88% on the subset of 8 lines (Table 4). Although transformation frequency did not differ between sublines selected for MV0103 and sublines selected for MV0110, a limited number of replications were performed. Additional replications may be performed to elucidate the particular haplotype.

TABLE 4
Transformation Performance of Selected Sublines from MV0040

| | | | | | | No. R0 Plants |
|---|---|---|---|---|---|---|
| Soybean | | No. Explants at Day | Total No. Rooted | Transformation | No. R0 Plants | with Backbone |
| Lines | Haplotype | 17 transfer | Shoots | Frequency | Died | (oriV PCR+) |
| 10032-1 | MV0103 | 1925 | 34 | 1.77% | 2 | 4 |
| 9910-1 | MV0110 | 1961 | 46 | 2.35% | 3 | 9 |
| 10041-1 | MV0103 | 2412 | 60 | 2.49% | 6 | 11 |
| 9991-1 | MV0110 | 2286 | 62 | 2.71% | 3 | 20 |
| 9990-1 | MV0103 | 2215 | 47 | 2.12% | 10 | 15 |
| 10084-1 | MV0110 | 2337 | 49 | 2.10% | 14 | 10 |
| 9995-1 | MV0103 | 2375 | 41 | 1.73% | 3 | 10 |
| 10010-1 | MV0110 | 2325 | 32 | 1.38% | 9 | 11 |
| MV0103 | — | 1325 | 3 | 0.23% | 1 | 0 |
| MV0110 | — | 1838 | 53 | 2.88% | 4 | 10 |

| | | No. R0 Plants | | | | No. R0 Plants 4 |
|---|---|---|---|---|---|---|
| | | possibly Epidermal | No. R0 Plants 1 | No. R0 Plants 2 | No. R0 Plants 3 | or more copies |
| Soybean | | (0 copies Invader | copy CP4syn | copies CP4syn | copies CP4syn | CP4syn |
| Lines | Haplotype | CP4syn) | (Invader) | (Invader) | (Invader) | (Invader) |
| 10032-1 | MV0103 | 2 | 10 | 17 | 0 | 5 |
| 9910-1 | MV0110 | 1 | 3 | 21 | 7 | 6 |
| 10041-1 | MV0103 | 4 | 12 | 26 | 5 | 7 |
| 9991-1 | MV0110 | 1 | 7 | 22 | 9 | 11 |
| 9990-1 | MV0103 | 6 | 13 | 20 | 3 | 3 |
| 10084-1 | MV0110 | 7 | 10 | 14 | 7 | 5 |
| 9995-1 | MV0103 | 4 | 10 | 16 | 6 | 5 |
| 10010-1 | MV0110 | 1 | 4 | 16 | 3 | 3 |
| MV0103 | — | 0 | 1 | 1 | 0 | 0 |
| MV0110 | — | 2 | 12 | 21 | 9 | 5 |

### Example 5

### Evaluating Soybean Sublines for Improved Transformation Performance

As mentioned earlier, MV0040 was developed from the cross of MV0110/MV0103. MV0040 has improved transformation frequency compared to MV0103, likely due to one or more genomic regions contributed by MV0110. Thirty percent of the 23 genomic regions were still segregating in the original MV0040 selection. Therefore, loci associated with transformation efficiency may be still segregated.

MV0040 sublines differing in the entirety of the segregating loci (one with all MV0103 alleles versus one with all MV0110 alleles) were evaluated for transformation performance. The following table shows the 8 segregating regions with their respective scores for MV0103 and MV0110, and the scores of the sublines that are closest to either parent across those regions (Table 5). Transformation frequency was slightly higher for sublines with haplotypes from parent MV0110 compared to sublines with haplotypes from parent MV0103 or heterozygosis in the regions, but did significantly differ (Table 6). Additional replications can be conducted to elucidate the particular haplotype.

TABLE 5
Examples of Segregating Regions in Sublines of MV0040

| Marker | NS0099457 | NS0097078 | NS0102044 | NS0100939 | NS0119353 | NS0116504 | NS0098306 | NS0103073 |
|---|---|---|---|---|---|---|---|---|
| MV0103 | AA | DD | GG | CC | TT | TT | AA | TT |
| MV0040 subline 10032-1 | AA | DD | GG | CC | TT | TT | AA | TT |
| MV0040 subline 10038-1 | AA | DD | GG | CC | TT | TT | AA | TT |
| MV0040 subline 10041-1 | AA | DD | GG | CC | TT | TT | AA | TT |
| MV0040 subline 9806-1 | AA | DD | GG | CC | TT | TT | AA | TT |
| MV0040 subline 9864-1 | AA | DD | GG | CC | TT | TT | AA | TT |
| MV0040 subline 9885-1 | AA | DD | GG | CC | TT | TT | AA | TT |
| MV0040 subline 9990-1 | AA | DD | GG | CC | TT | TT | AA | TT |
| MV0040 subline 9995-1 | AA | DD | GG | CC | TT | TT | AA | TT |
| MV0040 subline 10010-1 | AA | II | AA | TT | CC | AA | CC | AA |
| MV0040 subline 10084-1 | AG | II | AA | TT | CC | AA | CC | AA |
| MV0040 subline 9991-1 | AG | II | AA | TT | CC | AA | CC | AA |
| MV0040 subline 9910-1 | GG | II | AA | TT | CC | AA | CC | AA |
| >MV0110 | AG | II | AA | TT | CC | AA | CC | AA |

| Marker | NS0113949 | NS0100652 | NS0099639 | NS0124584 | NS0095258 | NS0124762 | NS0125528 | NS0119106 |
|---|---|---|---|---|---|---|---|---|
| MV0103 | AA | AA | TT | TT | CC | AA | CC | AA |
| MV0040 subline 10032-1 | AA | AA | TT | TT | CC | AA | CC | AA |
| MV0040 subline 10038-1 | AA | AA | TT | TT | CC | AA | CC | AA |
| MV0040 subline 10041-1 | AA | AA | TT | TT | CC | AA | CC | AA |
| MV0040 subline 9806-1 | AA | AA | TT | TT | CC | AA | CC | AA |
| MV0040 subline 9864-1 | AA | AA | TT | TT | CC | AA | CC | AA |
| MV0040 subline 9885-1 | AA | AA | TT | TT | CC | AA | CC | AA |
| MV0040 subline 9990-1 | AA | AA | TT | TT | CC | AA | CC | AA |
| MV0040 subline 9995-1 | AA | AA | TT | TT | CC | AA | CC | AA |
| MV0040 subline 10010-1 | TT | TT | CC | AA | TT | GG | GG | GG |
| MV0040 subline 10084-1 | TT | TT | CC | AA | TT | GG | GG | GG |
| MV0040 subline 9991-1 | TT | TT | CC | AA | TT | GG | GG | GG |
| MV0040 subline 9910-1 | TT | TT | CC | AA | TT | GG | GG | GG |
| MV0110 | TT | TT | CC | AA | TT | GG | GG | GG |

| Marker | NS0116502 | NS0119245 | NS0120228 | NS0119813 | NS0095368 | NS0093934 | NS0124203 |
|---|---|---|---|---|---|---|---|
| MV0103 | TT | TT | GG | AA | AA | TT | AA |
| MV0040 subline 10032-1 | TT | TT | GG | AA | AA | TT | AA |
| MV0040 subline 10038-1 | TT | TT | GG | AA | AA | TT | AA |
| MV0040 subline 10041-1 | TT | TT | GG | AA | AA | TT | AA |
| MV0040 subline 9806-1 | TT | TT | GG | AA | AA | TT | AA |
| MV0040 subline 9864-1 | TT | TT | GG | AA | AA | TT | AA |
| MV0040 subline 9885-1 | TT | TT | GG | AA | AA | TT | AA |
| MV0040 subline 9990-1 | TT | TT | GG | AA | AA | TT | AA |
| MV0040 subline 9995-1 | TT | TT | GG | AA | AA | TT | AA |
| MV0040 subline 10010-1 | CC | CC | AA | GG | GG | AA | TT |
| MV0040 subline 10084-1 | CC | CC | AA | GG | GG | AA | TT |
| MV0040 subline 9991-1 | CC | CC | AA | GG | GG | AA | TT |
| MV0040 subline 9910-1 | CC | CC | AA | GG | GG | AA | TT |
| MV0110 | CC | CC | AA | GG | GG | AA | TT |

TABLE 6
Transformation Frequency Based on
Similarity of Haplotype to Parent

| | # | # R0 Plants | Transformation |
|---|---|---|---|
| | Explants | Produced | Frequency |
| MV0110 | 1838 | 53 | 2.88% |
| MV0040(MV0110 | 8909 | 189 | 2.12% |
| haplotypes) | | | |
| MV0040 (MV0103 | 8927 | 182 | 2.04% |
| haplotypes) | | | |
| MV0040 (heterogeneous) | 3090 | 55 | 1.78% |
| MV0103 | 1325 | 3 | 0.23% |

MV0040 and MV0112 were evaluated for transformation performance with the same transformation construct. 11025 explants were evaluated for MV0040 and 11875 explants were evaluated for MV0112 (Table 7). MV0112 had a better recovery rate, shooting frequency and transformation frequency (TF) than the mother line MV0040. In fact, the transformation frequency of MV0112 was about 78% higher than the transformation frequency of the mother line MV0040. It is within the scope of the invention to evaluate the residual heterogeneity between the MV0040 and MV0112 for loci associated with transformation performance. Furthermore, reducing the heterogeneity within the transformation line may also increase the ability to select events with consistent expression levels. Additionally, a plurality of seed may be non-destructively sampled for DNA to assess the state of the heterogeneous loci. For example, U.S. Pat. Nos. 7,454,989; 7,713,351, and 6,959,617, incorporated herein by reference in its entirety, illustrate methods for non-destructive sampling of seed. Subsequently, seed may be bulked based on the state of the heterogeneous loci.

TABLE 7
Transformation Frequency (TF) of MV0040 (Mother line) and MV0112 (Subline)

| | | | | | | Cons | |
|---|---|---|---|---|---|---|---|
| | | | Explants | % | Shoots | Harvest | Cons Not |
| Cultivar | Explants | Discards | Calculated | Recovery | Harvested | From | Harvested |
| MV0040 | 11025 | 24 | 11625 | 27.5% | 468 | 440 | 0 |
| MV0112 | 11875 | 44 | 12975 | 30.7% | 937 | 355 | 84 |

| | Explants | | | Rooted | Total | | |
|---|---|---|---|---|---|---|---|
| | Harvested | Shooting | Rooted | Events | Rooted | Rooting | |
| Cultivar | From | Freq. | Events | Discarded | Events | Freq. | TF |
| MV0040 | 11000 | 4.3% | 150 | 55 | 205 | 43.8% | 1.86% |
| MV0112 | 8875 | 10.6% | 150 | 144 | 294 | 31.4% | 3.31% |

### Example 6

### Evaluating Corn Sublines for Improved Transformation Frequency

As with soybean, corn sublines that are isogenic for specific genomic regions may lead to an improved transformation system. Transformation using highly homogeneous plant material may eliminate potential variability between events due to heterogeneous genetic backgrounds. In addition, the residual genetic variability may be mined for improvements over the mother lines, such as transformation performance, yield and other traits.

Populations were developed from backcross of CV184 and CV185 to introgress transformation performance into an elite background. Individuals were subsequently self-pollinated after the BC3 generation in order to fix genetic regions. The population development is further described in Table 8.

TABLE 8
Population Development of Corn Sublines

| Cross | Generation | Progeny Screened | Activity |
|---|---|---|---|
| CV185 × CV184 | F1 | None | Cross transformability/culturability from CV184 into CV185 |
| (CV185 × CV184) × | BC1 | 279 | Backcross F1 to recurrent parent, CV185. Select for cultures that |
| CV185 | | | regenerate plants. Perform full genome scan and with |
| | | | culturability/transformability analysis to discover RFLP markers |
| | | | associated segregation distortion |
| Backcross to CV185 | BC2 | 300 | Use markers to select culturability/transformability regions and |
| | | | enrich elite regions |
| Backcross to CV185 | BC3 | 300 | Use markers to select culturability/transformability regions and |
| | | | enrich elite regions |
| BC3 × BC3 | F1 | 90 | Combine lines with highest number of |
| | | | culturability/transformability regions |
| Self | F2-F4 | 562, 357, 144 | Fix selected regions, test for and select transformable lines |

At the F4 generation, 144 individuals were genotyped with 172 SNP markers and compared to CV185 and CV184 (Table 9). Twenty-eight SNP loci were fixed across the 144 individuals. Nine SNP loci were still segregating within the F4 populations. The loci were located on chromosomes 1, 3, 4, 5, and 10. Twenty-eight of the 144 individuals were fixed at the additional 9 marker locations. In addition, sublines 65, 4, 99, 124, 134 were advanced due to similarity of haplotypes to the elite parent CV185. Furthermore, individuals may be phenotyped for a trait such as transformation performance, yield, and pest resistance. The phenotyping is correlated with genotyping to identify import loci and/or haplotypes. Furthermore, the subline populations can be selected and bulked based on the heterozygous regions. Individuals may be bulked with similar favorable haplotypes for such traits as transformation performance, yield or pest resistance. Additionally, individuals may be bulked with similar homozygous regions to generate a more uniform population.

TABLE 9
Genotypes of Parents (CV185 and CV184) and 144 F4 Individuals

| Marker | NC0008996 | NC0013490 | NC0030840 | NC0005177 | NC0108630 | NC0009523 | NC0012340 | NC0104957 | NC0004808 |
|---|---|---|---|---|---|---|---|---|---|
| Chromosome | 1 | 1 | 1 | 1 | 3 | 4 | 4 | 4 | 5 |
| Position | 189 | 198 | 198 | 216 | 123 | 1 | 1 | 4 | 12 |
| Region | 10 | 10 | 10 | 10 | 11 | 12 | 12 | 12 | 13 |
| Parents | | | | | | | | | |
| CV185 | AA | AA | AA | BB | BB | BB | BB | AA | BB |
| CV184 | BB | BB | BB | AA | AA | AA | AA | BB | AA |
| Subline No. | | | | | | | | | |
| 65 | AA | AA | AA | BB | AA | AA | AA | BB | BB |
| 4 | AA | AA | AA | BB | AA | AA | AA | BB | BB |
| 99 | AA | AA | AA | BB | AA | AA | AA | BB | BB |
| 124 | AA | AA | AA | BB | AA | AA | AA | BB | BB |
| 134 | AA | AA | AA | BB | AA | AA | AA | BB | BB |
| 1 | BB | BB | BB | BB | AA | AA | AA | BB | BB |
| 89 | BB | BB | BB | AA | BB | AA | AA | BB | BB |
| 25 | AA | AA | AA | BB | AA | AA | AA | BB | BB |
| 41 | BB | AA | AA | AA | BB | AA | AA | BB | BB |
| 42 | BB | BB | BB | BB | AA | AA | AA | BB | BB |
| 50 | BB | AA | AA | BB | AA | AA | AA | BB | BB |
| 19 | BB | BB | BB | BB | AA | AA | AA | BB | BB |
| 35 | AA | AA | AA | BB | AA | AA | AA | BB | BB |
| 59 | BB | BB | BB | AA | AA | AA | AA | BB | BB |
| 67 | BB | AA | AA | BB | AA | AA | AA | BB | BB |
| 68 | AA | AA | AA | BB | AA | AA | AA | BB | BB |
| 5 | BB | AA | AA | BB | BB | AA | AA | BB | BB |
| 29 | AA | AA | AA | BB | AA | AA | AA | BB | BB |
| 6 | BB | BB | BB | BB | AA | AA | AA | BB | BB |
| 80 | BB | AA | AA | BB | BB | AA | AA | BB | BB |
| 88 | BB | BB | BB | BB | AA | AA | AA | BB | BB |
| 16 | BB | BB | BB | BB | AA | AA | AA | BB | BB |
| 32 | BB | BB | BB | BB | AA | AA | AA | BB | BB |
| 48 | AA | AA | AA | BB | AA | AA | AA | BB | BB |
| 100 | BB | BB | BB | AA | BB | AA | AA | BB | BB |
| 103 | AA | AA | AA | BB | AA | AA | AA | BB | BB |
| 127 | BB | BB | BB | BB | AA | AA | AA | BB | BB |
| 104 | BB | BB | BB | BB | AA | AA | AA | BB | BB |
| 73 | BB | AB | AB | BB | AB | AA | AA | BB | BB |
| 81 | AA | AA | AA | BB | AA | AA | AA | BB | BB |
| 9 | AB | AB | AB | BB | AA | AA | AA | BB | BB |
| 17 | BB | AB | AB | AB | AB | AA | AA | BB | BB |
| 33 | AB | AB | AB | BB | AA | AA | AA | BB | BB |
| 49 | AB | AB | AB | BB | AA | AA | AA | BB | BB |
| 57 | BB | AB | AB | BB | AA | AA | AA | BB | BB |
| 2 | AB | AB | AB | BB | AA | AA | AA | BB | BB |
| 74 | AB | AB | AB | BB | AA | AA | AA | BB | BB |
| 82 | BB | AB | AB | AB | AB | AA | AA | BB | BB |
| 90 | BB | AB | AB | AB | AB | AA | AA | BB | BB |
| 10 | AB | AB | AB | BB | AA | AA | AA | BB | BB |
| 18 | BB | AB | AB | AB | AA | AA | AA | BB | BB |
| 26 | BB | BB | BB | AB | AB | AA | AA | BB | BB |
| 34 | AB | AB | AB | BB | AA | AA | AA | BB | BB |
| 58 | AA | AA | AA | BB | AA | AA | AA | BB | BB |
| 66 | BB | AB | AB | AB | AB | AA | AA | BB | BB |
| 3 | BB | AB | AB | AA | BB | AA | AA | BB | BB |
| 75 | AB | AB | AB | BB | AA | AA | AA | BB | BB |
| 83 | BB | BB | ** | BB | AA | AA | AA | BB | BB |
| 91 | BB | AA | AA | BB | AB | AA | AA | BB | BB |
| 11 | BB | BB | BB | AB | AB | AA | AA | BB | BB |
| 27 | AA | AA | AA | BB | AA | AA | AA | BB | BB |
| 43 | BB | AA | AA | AB | AB | AA | AA | BB | BB |
| 51 | BB | BB | BB | AB | AB | AA | AA | BB | BB |
| 76 | AB | AB | AB | BB | AA | AA | AA | BB | BB |
| 84 | AB | AB | AB | BB | AA | AA | AA | BB | BB |
| 92 | AB | AA | AA | BB | AA | AA | AA | BB | BB |
| 12 | AB | AA | AA | BB | AA | AA | AA | BB | BB |
| 20 | AA | AA | AA | BB | AA | AA | AA | BB | BB |
| 28 | AA | AA | AA | BB | AA | AA | AA | BB | BB |
| 36 | BB | BB | BB | AA | AB | AA | AA | BB | BB |
| 44 | AB | AB | AB | BB | AA | AA | AA | BB | BB |
| 52 | AB | AB | AB | BB | AA | AA | AA | BB | BB |
| 60 | AB | AB | AB | BB | AA | AA | AA | BB | BB |
| 77 | BB | AB | AB | AB | AB | AA | AA | BB | BB |
| 85 | AB | AA | AA | BB | AA | AA | AA | BB | BB |
| 93 | BB | AB | AB | BB | AA | AA | AA | BB | BB |
| 13 | BB | BB | BB | AB | BB | AA | AA | BB | BB |
| 21 | AB | AB | AB | BB | AA | AA | AA | BB | BB |
| 37 | AB | AB | AB | BB | AA | AA | AA | BB | BB |
| 45 | BB | AB | AB | BB | AA | AA | AA | BB | BB |
| 53 | AB | AA | AB | BB | AA | AA | AA | BB | BB |
| 61 | AB | AB | AB | BB | AA | AA | AA | BB | BB |
| 69 | AB | AB | AB | BB | AA | AA | AA | BB | BB |
| 78 | AB | AB | AB | BB | AA | AA | AA | BB | BB |
| 86 | AB | AB | AB | BB | AA | AA | AA | BB | BB |
| 94 | AB | AB | AB | BB | AA | AA | AA | BB | BB |
| 14 | BB | AB | AB | AB | BB | AA | AA | BB | BB |
| 22 | BB | AB | AB | BB | BB | AA | AA | BB | BB |
| 30 | BB | AB | AB | AB | BB | AA | AA | BB | BB |
| 38 | BB | BB | BB | BB | AA | AA | AA | BB | BB |
| 46 | AB | AB | AB | BB | AA | AA | AA | BB | BB |
| 54 | BB | BB | BB | BB | AA | AA | AA | BB | BB |
| 62 | BB | BB | BB | BB | AA | AA | AA | BB | BB |
| 70 | AB | AB | AB | BB | AA | AA | AA | BB | BB |
| 7 | AB | AB | AB | BB | AA | AA | AA | BB | BB |
| 79 | BB | AB | AB | AB | AB | AA | AA | BB | BB |
| 87 | BB | AB | AB | AB | AB | AA | AA | BB | BB |
| 95 | BB | AB | AB | AB | AA | AA | AA | BB | BB |
| 15 | BB | AB | AB | AB | AA | AA | AA | BB | BB |
| 23 | AB | AB | AB | BB | AA | AA | AA | BB | BB |
| 31 | AB | AB | AB | BB | AA | AA | AA | BB | BB |
| 39 | AB | AB | AB | BB | AA | AA | AA | BB | BB |
| 47 | AA | AA | AA | BB | AA | AA | AA | BB | BB |
| 55 | BB | AA | AA | BB | AB | AA | AA | BB | BB |
| 63 | BB | AB | AB | AB | AB | AA | AA | BB | BB |
| 71 | BB | AB | AB | AA | BB | AA | AA | BB | BB |
| 8 | AB | AB | AB | BB | AA | AA | AA | BB | BB |
| 96 | AB | AA | AA | BB | AA | AA | AA | BB | BB |
| 24 | BB | AB | AB | AA | AA | AA | AA | BB | BB |
| 40 | BB | BB | BB | BB | AA | AA | AA | BB | BB |
| 56 | BB | BB | BB | BB | AA | AA | AA | BB | BB |
| 64 | AB | AB | AB | BB | AA | AA | AA | BB | BB |
| 72 | BB | BB | BB | AA | AB | AA | AA | BB | BB |
| 97 | AB | AB | AB | BB | AA | AA | AA | BB | BB |
| 105 | BB | AB | AB | AB | AA | AA | AA | BB | BB |
| 113 | BB | BB | BB | BB | AA | AA | AA | BB | BB |
| 121 | AA | AA | AA | BB | AA | AA | AA | BB | BB |
| 129 | BB | AA | AA | AB | AB | AA | AA | BB | BB |
| 137 | BB | AA | AA | AB | BB | AA | AA | BB | BB |
| 98 | AB | AB | AB | BB | AA | AA | AA | BB | BB |
| 106 | BB | AB | AB | AB | BB | AA | AA | BB | BB |
| 114 | BB | AB | AB | BB | AA | AA | AA | BB | BB |
| 122 | AB | AB | AB | BB | AA | AA | AA | BB | BB |
| 130 | BB | AB | AB | AA | AB | AA | AA | BB | BB |
| 138 | AB | AB | AB | BB | AA | AA | AA | BB | BB |
| 107 | BB | BB | BB | BB | AA | AA | AA | BB | BB |
| 115 | BB | AA | AA | BB | AB | AA | AA | BB | BB |
| 123 | BB | AA | AA | AA | AB | AA | AA | BB | BB |
| 131 | AB | AB | AB | BB | AA | AA | AA | BB | BB |
| 139 | AA | AA | AA | BB | AA | AA | AA | BB | BB |
| 108 | AB | AB | AB | AB | AB | AA | AA | BB | BB |
| 116 | AB | AB | AB | BB | AA | AA | AA | BB | BB |
| 132 | AB | AB | AB | BB | AA | AA | AA | BB | BB |
| 140 | AA | AB | AB | BB | AA | AA | AA | BB | BB |
| 101 | BB | AB | AB | AB | BB | AA | AA | BB | BB |
| 109 | BB | BB | BB | AB | AB | AA | AA | BB | BB |
| 117 | AB | AB | AB | BB | AB | AA | AA | BB | BB |
| 125 | AA | AA | AA | BB | AA | AA | AA | BB | BB |
| 133 | AA | AA | AA | BB | AA | AA | AA | BB | BB |
| 141 | BB | BB | BB | AA | AB | AA | AA | BB | BB |
| 102 | AB | AB | AB | BB | AA | AA | AA | BB | BB |
| 110 | AA | AA | AA | BB | AA | AA | AA | BB | ** |
| 118 | AB | BB | BB | AB | AA | AA | AA | BB | BB |
| 126 | AB | AB | AB | BB | AA | AA | AA | BB | BB |
| 142 | AB | AB | AB | BB | AA | AA | AA | BB | BB |
| 111 | AB | AB | AB | BB | AA | AA | AA | BB | BB |
| 119 | BB | BB | BB | AA | AB | AA | AA | BB | BB |
| 135 | AB | AB | AB | BB | AA | AA | AA | BB | BB |
| 143 | BB | AA | AB | BB | BB | AA | AA | BB | BB |
| 112 | AA | AA | AA | BB | AA | AA | AA | BB | BB |
| 120 | AB | AA | AA | BB | AA | AA | AA | BB | BB |
| 128 | AB | AB | AB | BB | AA | AA | AA | BB | BB |
| 136 | AB | AB | AB | BB | AA | AA | AA | BB | BB |
| 144 | BB | BB | BB | AA | AB | AA | AA | BB | BB |

### Example 7

### Reducing Residual Heterogeneity to Increase Efficiency of Event Selection

Performance of a transgenic event may alter with subsequent generations of self-pollination. The underlying difference in the performance of an event may be germplasm-specific and related to differences in transcript regulation and the stability of transcripts and proteins. It can be hypothesized that transcription regulation can be affected by slight alterations in nucleotide sequence within the genome, affecting expression of the construct. Reducing residual heterogeneity within the original transformation germplasm may reduce the variability associated with evaluating transgenic effects. Additionally, reducing variability would allow for increases in efficiency in evaluating individual transgenic events. The reduction of variability would arise from the lower probability of having different events segregate during testing thereby reducing experimental error.

### Example 8

### Evaluating Residual Heterogeneity for Identification of Regions Associated with Traits of Interest in Soybean

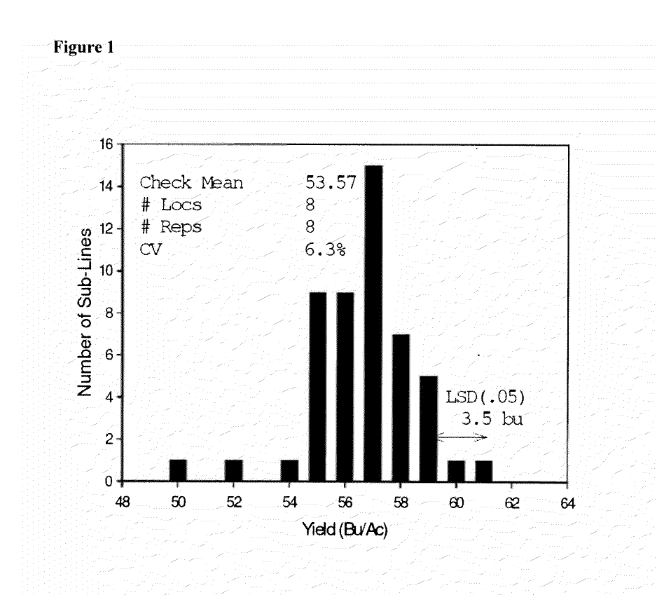

A subset of 48 F_{6:8}-derived sublines of MV0040 were fingerprinted with the 23 SNP markers listed in Tables 1-2. Yield tests were conducted on the 48 sublines in eight locations (FIG. 1). The mother line MV0040 had an average of 57 Bu/A. A number of sublines had significantly higher yield compared to the mother line. Moreover, four sublines had 3 Bu/A higher yield than the mother lines. Four QTLs associated with yield were identified using single marker analysis (Table 10).

TABLE 10
Molecular Markers Associated with Increased Yield

| | | Linkage | | | Yield | | Yield | |
|---|---|---|---|---|---|---|---|---|
| Marker | Region | Group | cM | Allele | (Bu/A) | Allele | (Bu/A) | P value |
| NS0100939 | 3 | B1 | 49.7 | T | 56.7 | C | 55.7 | 0.0178 |
| NS0119353 | 4 | B1 | 168.4 | T | 56 | C | 56.9 | 0.0884 |
| NS0116504 | 4 | B1 | 169 | T | 56.1 | A | 57.1 | 0.0565 |
| NS0098306 | 5 | G | 61.5 | C | 56.6 | A | 55.8 | 0.0912 |

A similar analysis can be performed to identify QTLs by taking advantage of the residual heterogeneity and phenotypic differences between sublines. QTLs identified from this analysis may include, but are not limited to yield, transfotinability, disease resistance, insect resistance, protein composition, oil composition, agronomic performance, and as stated in Example 3, selection for favorable alleles by employing a trait-haplotype association database predetermined by empirical methods (U.S. application Ser. No. 11/204,780), incorporated herein by reference in its entirety.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references.

What is claimed is:

1. A method for producing a transgenic plant comprising:
a. providing a substantially homozygous plant line suitable for transformation;
b. selecting a subline of the plant line having reduced heterogeneity;
c. transforming plant materials from the subline with a transgenic construct that confers a desired trait to at least one transformed plant;
d. recovering at least one transgenic event from the transformation step; and
e. selecting a transgenic event exhibiting a desirable level of the desired trait using plants of the subline as control.

2. The method of claim 1 wherein the subline has an increased transformation efficiency as compared to the substantially homozygous plant line.

3. The method of claim 1 wherein the subline has a transformation efficiency which is at least about 20% greater than the substantially homozygous plant line.

4. The method of claim 1 wherein the subline has a transformation efficiency which is at least about 50% greater than the substantially homozygous plant line.

5. The method of claim 1 wherein the subline has a transformation efficiency which is at least about 75% greater than the substantially homozygous plant line.

6. The method of claim 1 wherein the at least one transformed plant has a reduced variability between transgenic events as compared to a plant that is transformed directly from the substantially homozygous plant line.

7. The method of claim 1 wherein the at least one transformed plant has more consistent expression levels of the desired trait as compared to a plant that is transformed directly from the substantially homozygous plant line.

8. The method of claim 1 wherein the substantially homozygous plant line is an inbred plant line.

9. The method of claim 1 wherein the subline is selected using marker-assisted selection.

10. The method of claim 1 wherein the transformed subline is further self-pollinated to form a progeny line.

11. The method of claim 10 wherein marker-assisted selection is performed on the substantially homozygous plant line and the progeny line to identify individuals from the progeny line that exhibit reduced heterogeneity when compared with the substantially homozygous plant line.

12. The method of claim 1 wherein the desired trait is selected from the group consisting of herbicide tolerance, increased yield, insect control, fungal disease resistance, virus resistance, nematode resistance, bacterial disease resistance, abiotic stress tolerance, quality grain, mycoplasma disease resistance, modified oils production, high oil production, high protein production, germination and seedling growth control, enhanced animal and human nutrition, low raffinose levels, environmental stress resistance, increased digestibility, increased industrial enzymes, increased pharmaceutical proteins, increased peptides and small molecules, improved processing traits, improved flavor, nitrogen fixation, hybrid seed production, and/or reduced allergenicity, biopolymers, and biofuels.

13. The method of claim 12 wherein the herbicide for which the herbicide tolerance is created is selected from the group consisting of glyphosate, dicamba, glufosinate, sulfonylurea, bromoxynil and norflurazon herbicides.

14. The method of claim 1 wherein the substantially homozygous plant line is selected from the group consisting of maize, cotton, peanut, barley, oats, orchard grass, rice, sorghum, sugar cane, tall fescue, turfgrass species, wheat, alfalfa, members of the genus Brassica, broccoli, cabbage, carrot, cauliflower, Chinese cabbage, cucumber, dry bean, eggplant, fennel, garden beans, gourd, leek, lettuce, melon, okra, onion, pea, pepper, pumpkin, radish, spinach, squash, sweet corn, tomato, watermelon, ornamental plants, and other fruit, vegetable, tuber, oilseed, and root crops, wherein oilseed crops include soybean, canola, oil seed rape, oil palm, sunflower, olive, corn, cottonseed, peanut, flaxseed, safflower, and coconut.

15. The method of claim 1, wherein the substantially homozygous plant line is selected from the group consisting of soybean, corn, cotton, canola, pepper, and tomato.

16. The method of claim 1, wherein the substantially homozygous plant line is selected from the group consisting of Glycine arenaria, Glycine argyrea, Glycine canescens, Glycine clandestine, Glycine curvata, Glycine cyrtoloba, Glycine falcate, Glycine latifolia, Glycine latrobeana, Glycine max, Glycine microphylla, Glycine pescadrensis, Glycine pindanica, Glycine rubiginosa, Glycine soja, Glycine sp., Glycine stenophita, Glycine tabacina, and Glycine tomentella.

17. A transgenic plant produced according to the method of claim 1.

18. The plant of claim 17, wherein the transformation efficiency of the subline is at least equivalent to that of the substantially homozygous plant line.

19. The plant of claim 17, wherein the transformation efficiency of the subline is greater than that of the substantially homozygous plant line.

20. A transgenic plant produced according to the method comprising:
a. providing a substantially homozygous plant line suitable for transformation;
b. selecting a subline of the plant line having reduced heterogeneity;
c. transforming plant materials from the subline with a transgenic construct that confers a desired trait to at least one transformed plant;
d. recovering at least one transgenic event from the transformation step; and
e. selecting a transgenic event exhibiting a desirable level of the desired trait using plants of the subline as control, wherein the transformation efficiency of the subline is at least about 20% greater than the transformation efficiency of the substantially homozygous plant line.

21. A method for producing a plant having a desired trait comprising:
a. providing a substantially homozygous plant line;
b. selecting a subline of the plant line having reduced heterogeneity;
c. crossing at least one individual of the selected subline with a donor parent having at least one desired trait to form at least one second individual having the desired trait; and
d. backcrossing the at least one second individual with the at least one individual of the selected subline as a recurrent parent to form at least one progeny plant having the desired trait.

22. The method of claim 21 wherein the backcrossing step is repeated until the at least one progeny plant has a genotype that is substantially identical to that of the selected subline.

23. The method of claim 21 further comprising the step of self-fertilizing the at least one progeny plant.

24. The method of claim 21 wherein the selected subline does not contain the desired trait prior to the crossing step.

25. A plant produced according to the method of claim 21.


SEQUENCE LISTING (160 sequences)

SEQ ID NO: 1 - DNA, 626 bp, Glycine max
gtctgcctgcagccagctaggtatcaactttgttattgatcacttttcataattgtgggcatattctgaattttaaaaagtcaatctcctcaaattttcttaatcagatgaggcatttttttcttttatgaagccaacaacatatttgttgttccctacacctaaggttgtcaagaggcaggtgtgtagacagcaggttgaatggatcttctgtttgcacgtaagagttgaaaattttatggagtcctgttcgatcaaagttctggaatttgagtatgcctacaaaatccctctccaattctatgccagtcgaacttccactgctctgagatatggataagcaatatgctttcttatatcatagtatttgttattctgacacaatgcttttcgatgctctttcatttttctttttaatggcttggtgttggtataccccctacaataaaatgcaacttggggtgcaaattagcttatatatttgtaaattattcggttcagcttgtttaactaaataaataaagttaaacttttttttaaggataaagtaaacttatagtcacgcttgtttatttaaataaatcatgctcaattagtacttgacattattaagtcgtgcctaaagt

SEQ ID NO: 2 - DNA, 953 bp, Glycine max
  unsure (1)..(953): n = a, t, c, or g
cagcttgcatgcctgcagcgcctttgcatctctgcccacgaagaaatacaacatttcaccacaaatcaacgttacacacaattaatttaaaaaaaaaaaatgagattgcaaagtatgtgtggtaaatcacaacaacccaacctttccctgcgctgttcaggggtcaatccatcatctttctgcttgcctttagcaccagttctagcctgagccctttcgcgatcacgatccctctggctaccgcgtgcgctttagagttaattgaaaacaataacaaacaaaacggcttaacaaaccaaaccacgaataccccgtctgtattgtggcaaaggaaacaaaatttgaaaagaaaaggacaaaaaaaggtaaaattattaaaattgtgttattcttgtgtttgtttccatggatccaaactgaatgaaactattaattgcggtaaaaccaaaaagatctacaacaaatgggggtagcggagggtgaaatgtgatttatttttttcctcgaattttctaacagaaagtcaaccctatccctaaattaacgacaatttgtgaaaaattagcagtaaaatttcaacggatcaaaggatatgagtcattatcgtagatcgattattcgagaacgaaacgagcccggagacgaggggaaaccctaaaatatcaaaatcgggggaaataaaaaatacaattaacgaattgaaacanaaatcggggaaaagaacacatttgaagaaagaaataaccgaatggaaaaaggttctacctgttccaaggaaagaagagaaccagaatggatgctgcaaaaatgaaaacgttgaaaagaaaataaataaggttgaccatttaccgggacttaaaaaaattgttccccatggacatagacccccccataaaattaagatagagaactaaattttttttaatttaaaaaaaataaatttataggtattt

SEQ ID NO: 3 - DNA, 826 bp, Glycine max
  unsure (1)..(826): n = a, t, c, or g
aaggacaattaatgcacatcaattcactcaaatttggacttggagatgttcttcctttttttgaaccaagttacaaaacttgtttaacatttagttggttctatttacttgaaaaatcatcaactctctacgttattggttataacccatttatataaaatgataactacagaaaataggattttctaatagaaatttataaattatgtctgacatgcaaagattaataccacagaaaaatacaagttgcagtaaataactatcctgagggcatgcaaggtgcggaacctatcacaagtcacacacaagtcacaaatctgagcaaccctttgtaccaacagagctttatcacagtacacagatttgacagagtgggaaatcaaaacaaatcacatgaaagttaaaacacataataaccaaaaagttaagggactaagacctcacgaggtttaacaacaacggttagtaatgacaccaacaatagaaaaagcatgagaatcaaaagagcggtgactttcaagagagagtcatgataataaaaccctttcccttccacaacctcacatgactctcactccactgaaaaatgcaacaattttagcaaaacacaaacaatcacaagaaagtaaaaacgcataaacagaagaaattcagaaactaaaataaacaattcaaatgaaaagaaaataacataaaaaaataaaacaaacttatggaaattctcaaaatcattgcaacactggaatgcaacaccaaaaagttgactaaaattctattcaatattaacctttagcccctcctnctctgaaatatgtcattatgtgat

SEQ ID NO: 4 - DNA, 1530 bp, Glycine max
atcaataagaccttccctaccacccatggcatggaaaaagaactcttgaggggtaagtccacgtagatatgagttctccacaaagccacgactttcaggcccataatcatcttttgtgaagtgaggcaatgtacggtctatgaacccatatggaattcgcttaccctcaacgttttgttggcccacacaagcagtcatttgagaaatgttgataaaacttcctttggaaccagctgtcaccattgctttcagattgttgctctcagataaacttttttgagcactatttccagcatcatcacgagctctatttagagtctaaaattgtcaaatatgaaaattcgtcagatttacccaaaagaaaactttaataaatataacaatgtgacaagcaatccaaatgaaatacctggttcactctattttcaaatgaatccatcattgtccgaccaggttcagcctccaacttcttctcttgagcttccctaataagttgtttcactttctccttagctgctgaaatagtctgatttatggtttccatagtggaagcatcagcaattgtatcaccaattccaatgctaaaagcattctgcaaaagccagtagtttacaagccactgagtatgaccaagaaatttccgagcagcatcagggccaacctcttccctgtattcaagtagactttcagtgacgtaaacaaagaagaaaggaactgattgaaacagtttccagagccacaaagtttcacaactattatcagatagcactccataacattaaaatataaaattatttaacaaggttacttgacagttgaagtttggatcaatagaaaacaacccctctccttgggaagaggctgagctgtataggtagattgaccattacatgatccaattcatctagtaatttcaggtagaaatatttatataatttttattatactatatactgaagctgaaggaaagcattggtgcaactgttagagttgtttccatgtgactaagatgtcacatgttttaattgtggaatcaacctcttgcaaatgaaaggtaagtcagcctacaatagacctgtaatgggtccaacccttccctagaccatgtgatagttatagcaccaagtagcccattatatactgaagctgcataccaaatcacatgaatgagacttccagtagatgttccaagcgtctttttgcaaagtgttccagtaagtagctcccccttttcaattctgaccatggtatccccaggggttatggatcccctttcactctcactgtgccagctagaatatctaattagatttatttgtttaggaatgatgagattgaaaacttgcttccctgtccacaatggttctggctttaatattgcaggagcaggaactttcccatcaaaatcttcccaccacatcaatatgttcataaaaacatcctggttaaaaaagaagtttttatcagagatctaaacagcttgtacaagaaccatgaggctgaactaaactgcaggcatgcaagtcatgg

SEQ ID NO: 5 - DNA, 695 bp, Glycine max
aagctataacttagaagaggctgacataattatcggaaacaattatcaaactgtaaatgtacagacactttcggagttggtatcaactgaagataactatggagctagacttttcgtgtgcttattagcacagctcttggaggaagaaggccgggaatatgatataatatgtgttctgaatgtcatggcaactatggttctcgtctgtccagctgcttgtgaacaattatctttgcatggaatagcgacaacaatcaggacttggtgttatttgagcaatagcttgtcgacaacaacatacatgtccatcttgattttggtttttaacactttgagttcagtgcatcctgaaacactatcggttgatcaaagctgggtagcgataaccatgaaggtgtgggtagctgaacttctatttattcacattgcatcccgtataaattcaatcatgatcatgattagagactctgatttttatgtctcagtttctgaaaggcaagaaataaattgatataatctaaaatacaaaaccaaatagagatgagatggaatgcaacttatgcaagtactattttctgatattacaaaaattctataattaaccaactacccctaaatttactagaaaacacaatgatttagattaatgattgttctaacaccacgttaccacttctgcaggcatgcaagcttgg

SEQ ID NO: 6 - DNA, 856 bp, Glycine max
agcaattggtatacaagtaatatattttagttgtaatctaggctgggccgttagtgtgcctcaattctgaatgatacatcttatactacaagttagttttcaactttctctcaagaatacgaggagggagcttctccaacaacatcaaccatcaggaaagcagccaaggaaagtggatgagaagatcatttccggaaaataaagtctggaaatgagttactgtttcaggtcagttaattaacacttgccacatgctcaagtcaaacaccatggaccttaaagtcaaacacataaagaaataatataaggcctcaacctaaaaattgcccccaccttacagcataaggaaatattatcaggcccacaaaaccttaactacaaccataataatgaattttataattattaacagcagaatttaaatattatagttttgacaaggcatgaaattattaaatatatcaatacttgtcaatagagggtaagtgtccaagcaatggtaaggttactaccttgtgacctacaagtcatgggttcaaatcctagaaatagcccctaggtttacatctacgcttctcggaccctacatggtgggagactcatgcacatgctggatgcatacaagtggcaaaataatccatatatctataacccaacctatcaatcgaaaattaaatctgatgcaatcaatacatttcagtaaacaaaagtaaatggattgtatggactgtgttattgtgattaatggatggataatgatccatccatttgtttctcaattcattgaattaaatcactgaaaatggatcaagtggtttttcttcctattgctactgaaagcccatttttccttttt

SEQ ID NO: 7 - DNA, 654 bp, Glycine max
attcttcttgctacaacatccacagatgggaaatgccgagtattctccaccttgataaaaggcgttgatgcaaagtatgataaaagcaactctgaatgtggctgacttttactttttcttttttatatcattcaagtttaaatgttgacttggcacagggattcaaaaaagggtacttcatcagattcaaaatttggagaggttcaattttaactcttattcctcatatgaattttttatatttataatctacttaatgtataataaattttcatacatgttgcaatagatacattgcttgtgaagcttgtttatggatgtattgttcacctgttttttgattgaaaagttgaaactaatattatttataaaaaaataactttgttaaggtcttgatgtctggatttatatatatcagcattttttagcatatcgatcaatgactgaatgtggctgaaagagtactcttgcgaatgtttgatattttagagttgaatgtttgatatttaattgaatgcttataattatttttattttttaatgtattcttattctctaccttagcttacagaaaaaagaaatcccaattttttgacaaatgtgccttttttaataaaaaaaagatgaatttttaaaggttttttattaaatccg

SEQ ID NO: 8 - DNA, 647 bp, Glycine max
ctgtttagtgaggcttttaagccaattgaagtgaatataaaagttcaaatcacatgatttgtgcactgtcttaatgtgaatgctaagccttaaaatttttacagagctgatcacacaaaacttgatcttgtttgtgtcttgacatacagcctttgagatttgggactaccatgacattactttcagtttgggatcataatggctagattattttgtacaatgtatacacaaacgagttgaaatagcattgaaattttgatctgataagagttttaccaacattttccttttctttttcaggtcctttgtactgtatacaatggagcatgtagataaaggaaaagaatcatcaaatagtgacaatgaagtagaagccacaaattctccaattatttgtggccttccagatgatatttctcttatgtgcctggcaagaattcctagaaaatatcattctgtcctgaagtgtgtctcaaagagatggagggacttaatttgcagtgaagagtggatctgttatcgccgaaagcacaaactcgatgaaacctggatttatgctttgtgcaaggacaaatcaaaggagattttctgttatgttttggatccaactgaccccataagatattggaagctcgttggtggtcttc

SEQ ID NO: 9 - DNA, 888 bp, Glycine max
aagatataatgggcctatgctctgtctgtgcttcaagctgaaaggcttgtgtgtataagggagcatgttagagatagaagccattctggggcttagatctagatgctttaagctctttggaagttagtccttaacatgtctcaacctctactagtaagtattaactacctgtgtttttgtctgataccaaccagttatgatatttattttctcggtgaattctctgctcctgtaatgtatttcttcagagcttttgggacctgttgagtgaagcatgatataataaattcataacactgaccacagcaatataatttttcaatctcatgcaaatgcattgaattagtgcaattcagcttttggcattataatgatctacagtggatgctatcctttcttggcagttggccctgcttattgtgtataatgtgcttaaaggatacttgcacactggaggagaagaagcttagagatccaaggcgtctgtatgcacctggccgactctatcacattgtggagcgaaagccatttaggtaaagtatgatactctcaataaaatttgtctgaacatttgttgatctcataggtcttagctatgacatcttattgaacttcctctaattaaaggaagacttttcactgtccttattgtactaactatgtaaagcaaaatcatgcattttaacaaaaatatatcacatattttgaaatggtccaacatagtagtgagttagctaattgtggaggacgataatgaatgtggtcaatgggagctgatttatcgttccattgagttagctaattgtggaggacgataatgaatgtggtcaagaggcattcagtagacattccacatgcatgtggttctccctggaagttttacacagc

SEQ ID NO: 10 - DNA, 754 bp, Glycine max
gagccagtataagacatttgtggtgatcgctctcgaccatgagaagcagaatgcaaagcattctttgacaaaaattcatccacaagtgtttgtaagtaattgataacatctttggtccaaacatcagagcgagacagctgaatcttgtcagcagccccagaagaaataccaacagaaccaggtcgaacctgaaaaagccaatcataatctacaaatattaaaaaatataaagcaattcatcataataggtaaatataccaacactgaagcatccctacctgattgaggtaagtaaccttgataaaccaggtggccctaagcaatggaacattattcctgataagaacctctaaaagtgatgtccttttataaccatgaggaacatgatcagccaaagagcgtaatcgcttgtgctgctgagataaaccctgtaataaaattttaactgagttactaaagatacattgaaacaccaagtaatgttcaaaaagtagtatttaaggattttacagccacaacaacctgtcagcatcaacaacaaaaaacctaaggccctattcggggtggctgagttttctgttttccgttttaaaatgctatttcaaaatggaaggtgttcggctaaaatggttgcgagttggatttttatctgtttttaaaacagttgtcaccccattttataaacaataaaaataggttttatgttttattattttaaggttacttctatccctacctcaacgatctacgctgc

SEQ ID NO: 11 - DNA, 697 bp, Glycine max
aagtcgcataattttgctatgaacagctttttgtcttagaataatactattttcaatcctgaatataaaaaacaattatctaattcatcaagatcaataaaagtaatcatattagttaatttatcataaattttaattatattccatgactatccatgattacttttcaaaagataatttttcaaccaataagcttaccttgttttattgatagctcaataaagacgccacttttatgggaaaatgaatgtaatcattagggataaaaaataaatttagcaagaaaaaattaccttttgtttctgatatatgggatggaagtggatggaagtagtataatgatactcatgccattggattatctatattgcacatgctaatttcaagctattggaaaatgtggaaaagagatggaattataggcatgcaactttaatcaattactattttctagggtgacaatgtgtttagctctaacccgaattgaaactattaaaaaaatagatcacgatgcatgaagatagaagatatatatatcatttgagtctttgtaatgcatgaatgctcatattttattacccattaaaaaatatgggttgagtggtatattgaattttgatttattttgtcaatcagattagattcgaaccaactatttagtataaaaaatacttttcaaagcatttcaaattttcaa

SEQ ID NO: 12 - DNA, 937 bp, Glycine max
aatgaaggctgcctggaagaatgcagaagcagatccccttggatcggaggctctgataccaacttgaaagataataatctgaatattattgtaattctgtctttcgaagagtgaatacaagacttatttacaagctgccaatgacctaattaagaaaagaaataaaatggaacaaaatctgtacaaataaaaaaaaaatcaaatctgtactaattacagaataaaatctgtgctacttatagaatgtatgacaacatgagtaatttgagccatgattggattagtttattttgaagaaataaattcacttttttcatcttgaaagagctttagaagaaaaagtgattcttttcccataaataatcccaaccaaacatgtactcaatctgttggcaacaaaaagcaatgattcacgcaacaaatcataatacaggtgcttaacttaatctcaaaatgctccattttattcaacaatgtatgttacagagaaagagagtgcagctgaaagctctgtaaaaagtggtggtaaagtaactttgaagtatgacaaataggagtcagtgtcttatttgataactgcatgcattatcatcaaataattctcgtgatctaatcaatcattattgaggagtttcataccttctcaatgcgtgcacgctccagttggaagataggtttgtgttctctagtcaaaacaatggccttcagctgtgtctggacaccatctgggtgattttgggatccattagttgaccgagctaatactgccttggcatctcctatatttgcaaccacaacctaacaaatactcatcaaacaggaaaggccaaaaacaaattacaactgactttaaagaaaacagaaacaaaccaagagctgaattcatcaaaagattgttcaacaaaatacccataattatactgcctatgatgtttat

SEQ ID NO: 13 - DNA, 939 bp, Glycine max
gaagtgctcatccaatggaaagatttacctgactttgaagccacatgggaatcggttgttgttatgaagcaacaatttcttgcatatcaccttaaggacaaggttgctctattggcggggagtattgttaagctacctgttaagcaagtgtatgaagggaggacccatggcaagtataagcagaagccagagcagctgccagaaccagggttaggtagctgattaagatcagttacagttagtagttagccttgtaactgtttaggacagttaaaggtagttagttagctagtctttagctataaatagagactaattgcctggaattactcattcttgggtaatcacggtggttggttagggaggctaggctctgatccctaatcatacgtttgtcccacaagattaataaaatgagagttttggctagttctatcacaagacaacaagtaaccattgaaaatgaccttatttaaaatggttaattgaaaattcggtttttgaatgataccccaagggtgtcaattgattcatgttattgacctttggagtgagatgagccctggcggtagttggtattattaagctaaaattaattctattataaaaatggtttaatatttgcaaaaaaaattatcacaccaagcttaatttggaagaactttaacattttattaacatttatggatttgttcattatggtaccaatatgcgacagttattgtatatttcttccaaataaaaaaattttatcccaaattacttttatgggtgggataaggcgaataaaacattatttgtctcctcatcccaaagatacaatgttctattagtgtcaatgggcacacttggccatcctggaaaaatcttaaattttttgcggctttacccaaaccctttgagaatgaaatacctataactttccaccttatataacat

SEQ ID NO: 14 - DNA, 976 bp, Glycine max
tccttatatatactacctattatctatatatctcaatcttgataattccttttttaaaattgtaataaagcatgtatgtggttgatgtggtagctctaagaagcctaaaacaacaattgaacaaattttggagttaatgtttctaacttaattgattatcataaattgaccagttattatctatttgtgtgatcaatactatcagatctcttctacctactataccaacagaacattgtaaccagttcatcctggaagtagttttacagtttacatttatttatctgagttttattgatgtgctaatttgcagttgccgacacattgctttggaaggataaaaagcaaactttcatcgcattgttagtcttgattgcaatatacttcaacttcattgcatcggaaaataccatcattagtgctctgacaaagcttctattgtttgcatcgatctttttattcattcatgccattctacctgcaaaaatgtgagtacattatgtactgttgaatattgcagttgtcacacaattaacttgtccaatgataactataaagataatctagttgttagaagcattggagtttgtaatgttaaaataaatgctaattattgttctgctttaaatgcttgtgcagattggggtatactcttgagaaaatacccaaatcatggtttcacttatcagaagatttgtctcatcaaattgctctctcagtagcctcatcatggaatattgctgtgaatgttttgaaatcccttgcagaagggaatgattgggtgctgttcttcaaggtatctgcactgcattcgttttaaaatttctctatgaaattttgtttataagcaagcttacatatgcagacaatttactgagtataagtttctcaagtatcagaagtgctgacatctttcccttgctgatcaataggttgttctctctctgttcattctcagcttccttggagccttttc

SEQ ID NO: 15 - DNA, 1097 bp, Glycine max
agttgcacaaagattttatgctgttcttgaaattctatacgagctttctttcgttcaccgtgtttcagttatcatgtgtgcccacgaagaagacaagggcaattgtgtctgattttttcatcaatcacgcatgtgcacgtcacatgaccattttgggtttaacggaaataagtatgtaacaaaatggcaacaataatggatgtcattgtaataagtgttaaacacagggagacaactgtaatttggtctattctttttgctgaagaacctggtgtgtttttgttttttgtattgaaactattagacaacttagaggcccaaaattcatgagggttgggctttttgtacgtgggccttactggattcatgatccaaaccagccagatgtaccaaattcaatgttacaaatttctggtcctctattcattttaatgattgatagttacattttcagcaagctacccaaatttgtttgggtgcattttaaaagtcttaaggtcccacgttggcactttgcagagtggaccccaagaaaacatttgtcagaagacattttcagtacaaatggtcatatgattcaacttagttaaaaaaaaaatactattagtataaaaagatattttttttttaattcttcaaaagaaaaattatgaataatctaaagtttaacttaaccatcagtaaagtttgataaagaattattaatataatcaattataattagataatgagttaatttgtttaaacttaattttttaaaatatatatatatatatatttaaaaaaatgttttttaataaatagatatgatttatttcttaaaataattagaaaattatttattttatttaaataagaaattaaataagaaattatttatattttaagtaaaatcaatttaaaaaacatattagaaataataaaattttatttattttatttaaaaaataaatctaaacaaactagtccaatagtttattcacgtgtatgaaattggacgaaagtgtacgttgataattatctttttgggaaacgtagattttagtgatacttttctttttggtcaataacttagattttagttgaaatcagtagat

SEQ ID NO: 16 - DNA, 835 bp, Glycine max
ttgcatgcctgcagacatcctttgaaacacgctgtaaattgagaattgtatactgattattgaatcacctttgcagtcttacatcattgtcaatgattgtaccataactatttcatttcccattacttgtaataaaagtggctagtttatttttgaacatatgtattaataatagttgcacatgtgtgagatgatgatacatgtgcatcctgaactcttggaaaggtgctaaaatgagaaactatctttttaaatcaggctactttagttggttgaattctgaataaagtcctctaatttcttgttatgaattgatatatcctctaagaaataagaatatcgaattaaaagttgtttagagggaaaaagattccccagcttttaaatggacccagtttgttcaaatatcccatgcaacattttactctgaccatttcacttcaacccaagtaactaattgcatcatagtagcatcaaatacacaaacaaaaataagttaaaatcacttaatagaattagaagaaaaaaaaatcaaaatcaagattctagatattcattgccaaataaacaacgaactttgacagaagctgaaccagaaaagactaacaataactgcttaaaataaaatcatgccagacactgaattatgtggtcctattgattaactgaaagctccttccgcttatatcggaccatcactttccccttcactcccacaaccatggcattccaatcgatctctggaaaaggtgacaccaactccagctcaaaattcctaagcagatgagtccatattgcttttatctgcaggtcgactctagagaggacccgggtt

SEQ ID NO: 17 - DNA, 903 bp, Glycine max
gcttgcatgcctgcagctgatcccaacaatcagctctctcatcctgaatctctttctcagaatcaccaatttactaatttacacatgattcctgaagatcagctgcctcaacctgaatcgctgcccaactctgatccactgccgagttctgaaccaatgcaagacattcatctcacagacattaaaccgttacctcataatcatctggcccagtatgatacacttcccagcaatcatcatatggaccattctgaggcagtttccaaccatcaactaactcactctgagacactgtcccatgaacagctagccaattctcatttgttgcctcactatgggctgcaaactagtgaaacactacacgatcatccgatagttaattctcgaccacactatgagatagtcaacgctagcaacatacccagctatgagataataaatgctgaaactccattgaacagtgaggaacctactccagaaactcagccaagcaagaggaggaaaaagaagtctatagtctgggaacacttcaccatagaaacagtcagtcctggatgtagaagagcatgctgcatgcaatgcaagcaaagttttgcctacagcactggttcaaaagtcgctggtactagccaccttaaacgccacattgctaaggggacatgcccagctcttctacgcggccaagatcagaatcagttcagctcatatactccacgttcaaggggaagtgatgctgctggcaatgctagcagtgcacctaagcgacgttatagatcccctaatactccttacataatttttgatcaagatcgttgccgccatgagattgctagaatgatcattatgcatgattacccccttcacatggttgagcatccaggatttgttgcatttgtccaaaat

SEQ ID NO: 18 - DNA, 1450 bp, Glycine max
caagcttggcatgcctgcagaaaaatagtgagtgcaatgaatggcaagcttcagttcttcaggagcctaatgtggttaagaatgttcaccgttgggcttgtgggtaagctgttaactgaatctaaatgcttagcaagagttttattgggaggatcaattctccccaacataaagtttgttaatggggttgattaatataattgtaaggggacaagaccatgcccttacgtggatgttatctcgattaataagggcttggatcatactcactaattagtatacctaaatggcttctcttcaataatcataacactacaattacatccaattttgatttatctgtttatttttcctttacttgtgtacagacgtccgactgctttacaagatgttacatgacagggactatgatgttgcagctctagcaaattatttgagccaggcttttagataaaaaaaaattatgggaatgagaatgtctaacgaggagttttgtagttgttggcatgcttgctcatgtctgcttgtgtatttttgagtccctttcccctcaattatatgtctcccttatttttatttttatttttgggtcgggcactgcaacttaattgcatgctggactaatgttttgattgtcttggttgcaggaacgtggcaaccttgatcgatatgatgaggtagtaccgtagtaattttgtttactcttggtaactgttgtaaacttgtaatttaatgattgatcattgatgtcttgcccatcaaccaaaatatctagtatgatctgcagttttggaaataagatttttgtctgacaaccaagtgtttaaggtgtctatttatacttctactcataatcatagattagacccaaaatccaataactaatccaatcattaaaaaaccttgaaaataataaaagatcatgtgagtccattacaattctgaaaataagtccaaaatacaattaaaaaatgaaaataaatcttgttttaaaagttgactaaaattgattagttttcctttttttctattttttcatgagaaaatttagtctttcgtcaagctcttcttgaaatcatttcttacttgatcaagtgattaatctatccatgttagctcaccaaagtcaaattattcctccttgaatagtcttctatcattccctctctcttagaaagaatttgtcttcacaatgtcgaaggtatttacatcacaaagaatcaagtcagtaacattaaaaacgttgttttcaccatagtttagtggtaaatcaaggagatttatatcagtcgagtaaacatgcatgagttgttatatatctcctaacatcgacttcaatctccacaaataaaaaaaaatcattttcatcattaaaatcaatcttaaagattaaaatattcttatcatagaccgtctcgacaaaaggattaattattctgcaggtcgactc

SEQ ID NO: 19 - DNA, 728 bp, Glycine max
tttgtctctgaaatgttcaagtagcttctcaatactcataaccttatgttctgccaagtctctaactcgtacactcacctggaacaacaagacagcatgatcaataatcaaggagcaacagcgtaaaccctgaatgtctaggaacacaggcactataggaaaatagtaatgtcttcaaacatatcacagaccatagccacctctaagataatgtaaagcctccaaaattaatagatgatacggttggaatatggcaaaggaaaaaatgggtataagactgaagcataaaagggtaacaaactatgctgtaaaactgatcagactgaagcaaaaaacagaacagataataaatcagacagagcatcagattgaagaaaacaactatcagactgaagcggaaatagctatcagacggagcaacgatctaatcagactatgcaatcagactatgcagaagcataaaagtagtgaagtataaaccagaaaacataaacatatatgaagaagaagagagtaacaaaactgctttgatgtatttcaagtttataattcagttttgcacactatgaactgataaaataagtttttaaataggaaagctataaagccccctcattgggcaaaacatacagcaaattatgacaaagctagacatcattagccagcaactacaagttgctttccgagcacaagcaagctttaaacatagttgcaagcaaccacaaaat

SEQ ID NO: 20 - DNA, 730 bp, Glycine max
aagttaggatagtaatatccacaatactatcttgataaattgcattgaacacacatgcatgcacaaggtgtgtatatagtgaggaagaatcctgatactatattgtaaaaaaattcagtttattgtaaataaccatcagcattaatttttatattgaacatgcattttcctaaagtgcatgtaagacgctatacaacttcactgtgtatctccccacacaacttacactactaaaaataaaacaatgtatgcccccactttttttttttaggggaggggagtgcctaaactctcagcagtgaacttttttattcttattgtagcagcattcatccttcactgaagtaacagaggaacaaaaactgtataaaatatgcaagcttcaactctaagacaaattgaaggtgttagaaatgggggtctaccgttggccttgaaggatagtgaaaagatacatttagaaactcaatacgccctgttaacctctgcaatgttacccctgcaacaaagttacactcaaacttcaatatagtaaaattaaaacagaagcaaaaagttatgcaggaattcatcatgtttcccatgagtttctgttttctcttatttatttattgttacaatactgtaggtagaggaatgagaatagatacagatactggatacctagcactagctaataagtattactattatcacaaaaataataaattgagagctgcacatgacatga

SEQ ID NO: 21 - DNA, 774 bp, Glycine max
ttccggcaaggcttctgttgtgttctgttctacatctgtcttttttctttgttttctgttcagtcacaatggcttcatctggccagccttagaggcttaagataatgatgatgatgatatttgaatggatttaatatttagtagttgttcttagcctagaggcttatactttgtttgatttgatttgatggttttgtttattgtaattcagtaattgacaatttgacttgctgtttgctgttttgattatgcaactttatattttaatcagaatgtcttatatttaggtgtttttactttatttttcagtggtttgattagagttctttttcattttgcagcctcatttttacattccaaaataactaactgtttacttagctagttgcaacatctgatagtattatttattttgtggtttgtgctttggtggggttttagttctcataatttgttctcttcataatggtcgatctaaatcctaggccgatgtaaaaattttcttgttagcagagaatttcttatcttagtttgtagatatttgatgctccatcatgagtatttctgtgactgctaatttatacagtgatttttttttatgtagtgttataacttcctttcaacctatttgtgaagtttactccctcttttgtaaaactaatactagtgacttggcaggtaaaattgttcttggctgacgattacccctcaataggggtcagaatcacaagaacatcttcaggcaaagacatcttcaatgttacattctaaaat

SEQ ID NO: 22 - DNA, 524 bp, Glycine max
taccaccacaagtgcgtcctcaaagactgtggatatacccacttctgggctgaagcctgctattccatcagttctgttgtctacatctacacctgatgtgacagcttctgggactacacccacaagtgcagtgagttcattaccttctgggttttggttgaagccttcaaatccatcagtttcattattgaatcctacaccttgtaagacacccactctgtcagtgcccgcaattccatcattttcactattgtcttctacacatgatcagcctgtgactgcatcagttccattgtccaggcccgtatctggtaagccagtaactccctcagtttcattgcttacacatactcctgcgcttgatatgcacaggcctgtcagcaaaaattttcacgtgtcaagtggaatgccccctgctttaaatagccagtctcagcaagagacagctctggcatctgggccaaagcgagccgcatcattggcgtccataccttatagccaattaagctctggtccattgcctt

SEQ ID NO: 23 - DNA, 1159 bp, Glycine max
ttattgatggagtaagccgtatgtttgataatgtaaataagaagtacaatactaggctgtttggtggcttaataatgtctattatgggctatgtacacacaaaacctccaaaaagagagaattactggaagaaaaggaaagaatgcaaaaaccatttattattattgtgaagtgaagatcaattaaatagttatgatgacatagtatgagctcatatcttatgcttgtatgcagtgtcattgcaataataggtaaaatgacagctgtatatctttaaggatttctacttggagtgcatttgggtaaacaacataattatgcacttaagtggttatcacatgagggttacttgtaagctttactgtgaagcaactaaaataagttaataagaacatgttttgataagcttctgatttatagcttgttgaaagaaattaaaaagaacttataggaaggttatgagccagtttcataagctcaaataaaccttgtaggattttattttaccaaacctgccctaatgctacaaatgatttatggcatgtattaggtatgcactaaaaaaatattgtgcttggattcttcatataagtgctttatacttctaaattttcttttaagccttgatttctgttttttttttcctaagcattttgtttccgtatttctataccatttgcgatattaggtgcataaacttacgcatatctaggagtgaacgttatttacttgttagattcataaacttacacatatctaggagtgaacgttatttacttgttagattgggcttttaccaatcatgcctacttatgttaagttataactgatcatgattcgaaactattttttatttagcggtcaattctcccacgatggtagaattttattaatgggtatgtaatacaatgagtttttatctaatgcttgcacaagtacacaatcatagtgtcttattgacatgcactggggaaagacatgctttaccttgtttccatcctaaatataagccctagtttgtgtggcttgcttcatctgttttctgtttcatttatattatttgttgtcttgtttagagatggcacggcgtgagagacaaagaaggagtaggaggcagagatggatttggggatctattactactgccattgcagttggt

SEQ ID NO: 24 - DNA, 26 bp, Artificial Sequence
  Other information Description of artificial sequence synthetic primer
gcacgtaagagttgaaaattttatgg

SEQ ID NO: 25 - DNA, 23 bp, Artificial Sequence
  Other information Description of artificial sequence synthetic primer
tggagagggattttgtaggcata

SEQ ID NO: 26 - DNA, 20 bp, Artificial Sequence
  Other information Description of artificial sequence synthetic primer
gcatctctgcccacgaagaa

SEQ ID NO: 27 - DNA, 26 bp, Artificial Sequence
  Other information Description of artificial sequence synthetic primer
ccacacatactttgcaatctcatttt

SEQ ID NO: 28 - DNA, 27 bp, Artificial Sequence
  Other information Description of artificial sequence synthetic primer
accaacagagctttatcacagtacaca

SEQ ID NO: 29 - DNA, 28 bp, Artificial Sequence
  Other information Description of artificial sequence synthetic primer
aggtcttagtcccttaactttttggtta

SEQ ID NO: 30 - DNA, 27 bp, Artificial Sequence
  Other information Description of artificial sequence synthetic primer
actctcactgtgccagctagaatatct

SEQ ID NO: 31 - DNA, 25 bp, Artificial Sequence
  Other information Description of artificial sequence synthetic primer
ctgcaatattaaagccagaaccatt

SEQ ID NO: 32 - DNA, 30 bp, Artificial Sequence
  Other information Description of artificial sequence synthetic primer
tgcaacttatgcaagtactattttctgata

SEQ ID NO: 33 - DNA, 33 bp, Artificial Sequence
  Other information Description of artificial sequence synthetic primer
caatcattaatctaaatcattgtgttttctagt

SEQ ID NO: 34 - DNA, 27 bp, Artificial Sequence
  Other information Description of artificial sequence synthetic primer
ggaaatgagttactgtttcaggtcagt

SEQ ID NO: 35 - DNA, 23 bp, Artificial Sequence
  Other information Description of artificial sequence synthetic primer
aggtccatggtgtttgacttgag

SEQ ID NO: 36 - DNA, 27 bp, Artificial Sequence
  Other information Description of artificial sequence synthetic primer
gaagcttgtttatggatgtattgttca

SEQ ID NO: 37 - DNA, 27 bp, Artificial Sequence
  Other information Description of artificial sequence synthetic primer
tccagacatcaagaccttaacaaagtt

SEQ ID NO: 38 - DNA, 26 bp, Artificial Sequence
  Other information Description of artificial sequence synthetic primer
atttgtgcactgtcttaatgtgaatg

SEQ ID NO: 39 - DNA, 25 bp, Artificial Sequence
  Other information Description of artificial sequence synthetic primer
tcaagttttgtgtgatcagctctgt

SEQ ID NO: 40 - DNA, 27 bp, Artificial Sequence
  Other information Description of artificial sequence synthetic primer
ttttgaaatggtccaacatagtagtga

SEQ ID NO: 41 - DNA, 24 bp, Artificial Sequence
  Other information Description of artificial sequence synthetic primer
gctcccattgaccacattcattat

SEQ ID NO: 42 - DNA, 26 bp, Artificial Sequence
  Other information Description of artificial sequence synthetic primer
cctgaaaaagccaatcataatctaca

SEQ ID NO: 43 - DNA, 23 bp, Artificial Sequence
  Other information Description of artificial sequence synthetic primer
caggtagggatgcttcagtgttg

SEQ ID NO: 44 - DNA, 28 bp, Artificial Sequence
  Other information Description of artificial sequence synthetic primer
atggaagtggatggaagtagtataatga

SEQ ID NO: 45 - DNA, 24 bp, Artificial Sequence
  Other information Description of artificial sequence synthetic primer
tttccacattttccaatagcttga

SEQ ID NO: 46 - DNA, 22 bp, Artificial Sequence
  Other information Description of artificial sequence synthetic primer
tggcaacaaaaagcaatgattc

SEQ ID NO: 47 - DNA, 26 bp, Artificial Sequence
  Other information Description of artificial sequence synthetic primer
ggagcattttgagattaagttaagca

SEQ ID NO: 48 - DNA, 21 bp, Artificial Sequence
  Other information Description of artificial sequence synthetic primer
cacatgggaatcggttgttgt

SEQ ID NO: 49 - DNA, 21 bp, Artificial Sequence
  Other information Description of artificial sequence synthetic primer
cgccaatagagcaaccttgtc

SEQ ID NO: 50 - DNA, 30 bp, Artificial Sequence
  Other information Description of artificial sequence synthetic primer
tgaccagttattatctatttgtgtgatcaa

SEQ ID NO: 51 - DNA, 20 bp, Artificial Sequence
  Other information Description of artificial sequence synthetic primer
tcggcaactgcaaattagca

SEQ ID NO: 52 - DNA, 23 bp, Artificial Sequence
  Other information Description of artificial sequence synthetic primer
ttttttcatcaatcacgcatgtg

SEQ ID NO: 53 - DNA, 28 bp, Artificial Sequence
  Other information Description of artificial sequence synthetic primer
gttgccattttgttacatacttatttcc

SEQ ID NO: 54 - DNA, 25 bp, Artificial Sequence
  Other information Description of artificial sequence synthetic primer
cttttaaatggacccagtttgttca

SEQ ID NO: 55 - DNA, 23 bp, Artificial Sequence
  Other information Description of artificial sequence synthetic primer
tgggttgaagtgaaatggtcaga

SEQ ID NO: 56 - DNA, 25 bp, Artificial Sequence
  Other information Description of artificial sequence synthetic primer
ccgatagttaattctcgaccacact

SEQ ID NO: 57 - DNA, 28 bp, Artificial Sequence
  Other information Description of artificial sequence synthetic primer
ggagtttcagcatttattatctcatagc

SEQ ID NO: 58 - DNA, 26 bp, Artificial Sequence
  Other information Description of artificial sequence synthetic primer
agtcgagtaaacatgcatgagttgtt

SEQ ID NO: 59 - DNA, 28 bp, Artificial Sequence
  Other information Description of artificial sequence synthetic primer
gaaaatgatttttttttatttgtggaga

SEQ ID NO: 60 - DNA, 26 bp, Artificial Sequence
  Other information Description of artificial sequence synthetic primer
tctgccaagtctctaactcgtacact

SEQ ID NO: 61 - DNA, 23 bp, Artificial Sequence
  Other information Description of artificial sequence synthetic primer
acgctgttgctccttgattattg

SEQ ID NO: 62 - DNA, 33 bp, Artificial Sequence
  Other information Description of artificial sequence synthetic primer
aatactgtaggtagaggaatgagaatagataca

SEQ ID NO: 63 - DNA, 27 bp, Artificial Sequence
  Other information Description of artificial sequence synthetic primer
gcagctctcaatttattatttttgtga

SEQ ID NO: 64 - DNA, 19 bp, Artificial Sequence
  Other information Description of artificial sequence synthetic primer
ccggcaaggcttctgttgt

SEQ ID NO: 65 - DNA, 23 bp, Artificial Sequence
  Other information Description of artificial sequence synthetic primer
tgaagccattgtgactgaacaga

SEQ ID NO: 66 - DNA, 23 bp, Artificial Sequence
  Other information Description of artificial sequence synthetic primer
gcgtcctcaaagactgtggatat

SEQ ID NO: 67 - DNA, 27 bp, Artificial Sequence
  Other information Description of artificial sequence synthetic primer
gtagacaacagaactgatggaatagca

SEQ ID NO: 68 - DNA, 26 bp, Artificial Sequence
  Other information Description of artificial sequence synthetic primer
agtggttatcacatgagggttacttg

SEQ ID NO: 69 - DNA, 29 bp, Artificial Sequence
  Other information Description of artificial sequence synthetic primer
ttctttcaacaagctataaatcagaagct

SEQ ID NO: 70 - DNA, 14 bp, Artificial Sequence
  Other information Description of artificial sequence synthetic probe
cgatcgaagttctg

SEQ ID NO: 71 - DNA, 16 bp, Artificial Sequence
  Other information Description of artificial sequence synthetic probe
ttcgatcaaagttctg

SEQ ID NO: 72 - DNA, 15 bp, Artificial Sequence
  Other information Description of artificial sequence synthetic probe
ttgtggtgaaatgtt

SEQ ID NO: 73 - DNA, 14 bp, Artificial Sequence
  Other information Description of artificial sequence synthetic probe
tgtggtgaatgttg

SEQ ID NO: 74 - DNA, 14 bp, Artificial Sequence
  Other information Description of artificial sequence synthetic probe
agagtgggaaatca

SEQ ID NO: 75 - DNA, 16 bp, Artificial Sequence
  Other information Description of artificial sequence synthetic probe
agagtgagaaatcaaa

SEQ ID NO: 76 - DNA, 15 bp, Artificial Sequence
  Other information Description of artificial sequence synthetic probe
cagggaaacaagttt

SEQ ID NO: 77 - DNA, 14 bp, Artificial Sequence
  Other information Description of artificial sequence synthetic probe
agggaagcaagttt

SEQ ID NO: 78 - DNA, 20 bp, Artificial Sequence
  Other information Description of artificial sequence synthetic probe
tctataattaaccaactacc

SEQ ID NO: 79 - DNA, 20 bp, Artificial Sequence
  Other information Description of artificial sequence synthetic probe
tctataactaaccaactacc

SEQ ID NO: 80 - DNA, 14 bp, Artificial Sequence
  Other information Description of artificial sequence synthetic probe
catgtggcaagtgt

SEQ ID NO: 81 - DNA, 16 bp, Artificial Sequence
  Other information Description of artificial sequence synthetic probe
tgtggctagtgttaat

SEQ ID NO: 82 - DNA, 16 bp, Artificial Sequence
  Other information Description of artificial sequence synthetic probe
caacttgtcaatcaaa

SEQ ID NO: 83 - DNA, 18 bp, Artificial Sequence
  Other information Description of artificial sequence synthetic probe
ttcaacttttcaatcaaa

SEQ ID NO: 84 - DNA, 16 bp, Artificial Sequence
  Other information Description of artificial sequence synthetic probe
ctaagccttaaaattt

SEQ ID NO: 85 - DNA, 16 bp, Artificial Sequence
  Other information Description of artificial sequence synthetic probe
aagccttaaaaatttt

SEQ ID NO: 86 - DNA, 14 bp, Artificial Sequence
  Other information Description of artificial sequence synthetic probe
cgtcctccacaatt

SEQ ID NO: 87 - DNA, 15 bp, Artificial Sequence
  Other information Description of artificial sequence synthetic probe
cgtcctccactatta

SEQ ID NO: 88 - DNA, 19 bp, Artificial Sequence
  Other information Description of artificial sequence synthetic probe
catcataattggtaaatat

SEQ ID NO: 89 - DNA, 18 bp, Artificial Sequence
  Other information Description of artificial sequence synthetic probe
agcaattcatcataatag

SEQ ID NO: 90 - DNA, 18 bp, Artificial Sequence
  Other information Description of artificial sequence synthetic probe
atgtgcaatatagataat

SEQ ID NO: 91 - DNA, 18 bp, Artificial Sequence
  Other information Description of artificial sequence synthetic probe
tgcaatataggtaatcca

SEQ ID NO: 92 - DNA, 17 bp, Artificial Sequence
  Other information Description of artificial sequence synthetic probe
acgcaacatatcataat

SEQ ID NO: 93 - DNA, 13 bp, Artificial Sequence
  Other information Description of artificial sequence synthetic probe
cgcaacaaatcat

SEQ ID NO: 94 - DNA, 16 bp, Artificial Sequence
  Other information Description of artificial sequence synthetic probe
tgatatgcaagaaatt

SEQ ID NO: 95 - DNA, 14 bp, Artificial Sequence
  Other information Description of artificial sequence synthetic probe
atgcaggaaattgt

SEQ ID NO: 96 - DNA, 20 bp, Artificial Sequence
  Other information Description of artificial sequence synthetic probe
aaactgtaaaactacttcca

SEQ ID NO: 97 - DNA, 19 bp, Artificial Sequence
  Other information Description of artificial sequence synthetic probe
tgtaaactgtaaaattact

SEQ ID NO: 98 - DNA, 17 bp, Artificial Sequence
  Other information Description of artificial sequence synthetic probe
atgaccattttgggttt

SEQ ID NO: 99 - DNA, 17 bp, Artificial Sequence
  Other information Description of artificial sequence synthetic probe
tgaccattttcggttta

SEQ ID NO: 100 - DNA, 14 bp, Artificial Sequence
  Other information Description of artificial sequence synthetic probe
catgcagcatttta

SEQ ID NO: 101 - DNA, 13 bp, Artificial Sequence
  Other information Description of artificial sequence synthetic probe
cccatgcaacatt

SEQ ID NO: 102 - DNA, 17 bp, Artificial Sequence
  Other information Description of artificial sequence synthetic probe
agtcaatgctagcaaca

SEQ ID NO: 103 - DNA, 17 bp, Artificial Sequence
  Other information Description of artificial sequence synthetic probe
tagtcaacgctagcaac

SEQ ID NO: 104 - DNA, 17 bp, Artificial Sequence
  Other information Description of artificial sequence synthetic probe
cctaacattgacttcaa

SEQ ID NO: 105 - DNA, 16 bp, Artificial Sequence
  Other information Description of artificial sequence synthetic probe
cctaacatcgacttca

SEQ ID NO: 106 - DNA, 16 bp, Artificial Sequence
  Other information Description of artificial sequence synthetic probe
tgctgtcttgttgttc

SEQ ID NO: 107 - DNA, 16 bp, Artificial Sequence
  Other information Description of artificial sequence synthetic probe
tgctgttttgttgttc

SEQ ID NO: 108 - DNA, 15 bp, Artificial Sequence
  Other information Description of artificial sequence synthetic probe
tgctaggcatccagt

SEQ ID NO: 109 - DNA, 16 bp, Artificial Sequence
  Other information Description of artificial sequence synthetic probe
ctagtgctaggtatcc

SEQ ID NO: 110 - DNA, 17 bp, Artificial Sequence
  Other information Description of artificial sequence synthetic probe
ttctacgtctgtctttt

SEQ ID NO: 111 - DNA, 18 bp, Artificial Sequence
  Other information Description of artificial sequence synthetic probe
ttctacatctgtcttttt

SEQ ID NO: 112 - DNA, 13 bp, Artificial Sequence
  Other information Description of artificial sequence synthetic probe
ctgggctgaagcc

SEQ ID NO: 113 - DNA, 13 bp, Artificial Sequence
  Other information Description of artificial sequence synthetic probe
tctgggcagaagc

SEQ ID NO: 114 - DNA, 18 bp, Artificial Sequence
  Other information Description of artificial sequence synthetic probe
tatcaaaacaagttctta

SEQ ID NO: 115 - DNA, 20 bp, Artificial Sequence
  Other information Description of artificial sequence synthetic probe
tatcaaaacatgttcttatt

SEQ ID NO: 116 - DNA, 663 bp, Zea mays
  unsure (1)..(663): n = a, t, c, or g
cataatttcatgtactgttcgtacagagattatctttagagagaatagtaagtactaccttcgttcttgaatatttatcatctgctagtttaattttaaactaaaacgtgataaataaaaaaancgaagagggtatcttctatcttgtaataccaaccacgtgaagaccttcactccacngttgttttgcctttttagattatattaaaactttgcccatacaaaacagtttcttagggaaatttccagatttatctcactcgcaattaccaactggccatcgttatttttnagaacgatatcttgcaatcttctaagctgactctgtaaatcttcacgtanccatctcctaaaaatgaggctctagtttttatatttcatggcttcaactgaacaatcacggtcctcgttttttttaaaaaaaatgagaaaagtgtgtctttaaagtaacctatagccacaccatctagtatgccaaaaaaatggtggatttttcattggccgacaccgtagacctgctgcttaagtaaatatatctggtttggggttaattagnagtatgccactccaaatttaaagagttataaagtagtttattttcatgtcgctcnatgacatcaanatgaatnacctatctnnttttatnnaannnnnnnnnnnngt

SEQ ID NO: 117 - DNA, 619 bp, Zea mays
  unsure (1)..(619): n = a, t, c, or g
ctgcgtcgtcgtgttgttacgatcgtccttttttttttgaaaatttattgaaggcccgtgcttcttcttctcttcttcttttccttctccactgttttcctcgcatgctacatgacagtctctctctctcgttaactctgttggtgttcttattatttgatccggatgcaagtaatactatacaaacaggaatcgggaatgcacgctcccaatttttgacgcctccatgcatgtagtggnggagttccgtaaaattaacgtccgagatgtactattctatcttttgattttttttnncgtgttttatttaaaaataaacancggatgataaatatttaataacngagagtaacatttaaaacaatccagttaaactatgacaagtagggaaagtggattagatattcgggaacaattactnacaatagaggagaatcacgagcacatggcagcagacagtcaacactcaggacacaacgctcgccgctcaggtggncgttgttcattggccanggaggcactctgcactcgcctatgaataacaaaaaaagataatcaagttgagaaagttatatagcntggaagaagatgtaatgncnngggtggatttgggcgnnng

SEQ ID NO: 118 - DNA, 1285 bp, Zea mays
  unsure (1)..(1285): n = a, t, c, or g
catggactttggctaaaggacaagacaccttcaccccaattagtgcagtagtaagcaatgatttttcttccctgtgcttggagttggttggtggtggataaggacaatatgttttatttatttgtactcncttgttataattacacctcaatgaatcaatgccatantcaggttagtcatgcagaagtgttgttagatanaaacacttgatntttgttgccttccatgctctccgcaggcatctagtgacatgaaatgtgtaatcccntcattattacacaccatgttttttacttaagttaacaataatttctccatcaaccaggttccaaaatcagctgtcactaatcccgatgatctcgagctctggctgaaggtctctcaaatgtgtactgaatttgtttaatgttacctgaattgctagagcatatggtgtacaattgactaccaactgcatccaggtagatgatgaactaagncagaaaggaccgacaagcgacatgctattcaagattccttttctgataagctatatcagttccatcatgacattaatggangntgatgtgatactaacaggtagtgacatctnctctacgtgtagcttnnntttgcannnnnnnnnnnnnnnnnnnnnnnncatttgttcctcaacanntacnnnngngggtgtgggtcctgtccgagtaggtcagaagattacagctggtataactgacctaattgatgttgagttcgatgttcagaggcgcaatcggtcattttccacatgatgcaataagcaagaaagtaatggtaagcaacataagatagtaaaggtgcatgcttttatttgttgtgtgactgaaaataatatcaggcaattgatttcatataagcaaaacctgtttacccatagcaagaccagtctgatcagagagggcaccatcgtatccatgttcgaggacatccttgaagatgtactacaattggacagccagatggtgtatatgtatagggtatagggccaatatgccagaactgtgttatggggcatcatcataacaaattgtgctaaagctacgttgtcctactccacattctcacatatacatatcaatcaaaacctgaagtacatgaactacttatcagttagaagctgtgctgtatgaactattgttaccgtgatttctttaaaagaatatatgctattgttggattagttataatgatcttaacaatttcaatagtaattattgatgcctatgcgggattcagcgacttaattatttctggctcgtgctgt

SEQ ID NO: 119 - DNA, 668 bp, Zea mays
  unsure (1)..(668): n = a, t, c, or g
tccataatctctatgagcaatagactggcatatcgattcagtaatcaactgtacatcagggatctctcctctttcaagtatcgagtcaagtgcatgctccagcaaatatggctgcacataaaatcataaagagnnnnnnnnnnnnnnnnnnnnnncaattcctattttggtcacttctaatataaaaaaggacagacccagtgttggcactcccacttaacttttgcagagagtttatatcgaactcgaccnnnnnnnnnnnnnnnnnnnnnnnnnccacaactacatctggcatgaagttgtagctatttagcatatgtcctttcaattgaagtacacaaagtgtaaacataaccaaaaagttttcagtgcacatctctactacttattgagacgatattgtagaccatatgcacaaccatccaccctcgtcgtgttgttcgttcccggccgatgcgtgcagtgtcggcaatctcaccgnngccttgtcgcattgnnntcnggtctgctgtagatatgcctagtcgncctgtnttccttccctctactannnnnncatcggggagannnggagnacnnngcatncgnntggaannagatctatcgagttnntgtnnnnannnnnnnnnnntnnncatatgatacanncncnntttctgcacannnng

SEQ ID NO: 120 - DNA, 626 bp, Zea mays
  unsure (1)..(626): n = a, t, c, or g
cagttctgtgcaacatacaaaatttttacgcttactcaccatgtagatatgtttttttagtactcagtgcaatagcctataatgtattttttttnccaacctagaaacttgtggctatttgacatactgaatatgtgaaaccatactgactatgcaaaactatagtttcttccttagataagtgcaaaaactaacttgatattgtttcctgtggggattattatttattcattctggctagaagtttggggaatatatagtcacagatctttgatgtaccataacctttttcattcgcttggtctggactctagaaaacttttgggcattttgtgaagattgcctgcaggctaaacactctgtagggactgactgttagactgttgttttcttgtgttgttttcctgtctaaatgttatgggcctagagcccagcccatgtaatgtgatatatgggtttagggtttagggtttcacttctcgtaacagtgacgcctaatagggggcgggggaggtgaattatgggtatttgtttacttggcccagggtgcctgcatctgctgggctgggccaaggcaggatcggtcaggacttgccggtgcagggtctcttgtttggttgggtgga

SEQ ID NO: 121 - DNA, 566 bp, Zea mays
  unsure (1)..(566): n = a, t, c, or g
atggcgtacaagttctccaacctggccatctgcaaggccgtcggcggcggcgggaggaagaaggtgcacatcgtggactnnngcgaccactactacgggntccagtggccgtcgttgctgggctactggggtagcctggaggctgggccgcccgaggtgaggatcaccgccatcgacttccccgagcccgggttccgnccggatgctcggctccaggcgaccgggcggcggctcacatgcttcgctcgncggcatggcgtccccttgaggttccacggcatcgnggccaggtgggacgcggtttcggtggacgagctgagcatcgagcgcgacgaggtgctcgtcgtgaacggcctgttcagtctcggtaggatgcaggagcaggagcaggacgacgtggatagggatagccggccgagccctagggacacagtcctcggtaacgtccggaagatgcggcccgacgtgttcgtcctgtgcgtggagaacagctcgtacgnnncgcccttnnncgtgacgnnnnnnngggnnnnnctnnnnnactactnnnnnnnnnnnnnnnngat

SEQ ID NO: 122 - DNA, 655 bp, Zea mays
  unsure (1)..(655): n = a, t, c, or g
tgagcgatgaggatggtgcgctggtggacgcgctcaaccgtcgagctgctgtttctgccgccgcccccagcttcttcgaggggttcgcgctccacgggatccgcgtggagagcatccgcccaggccacatcctctgctccttcacggtccccgcacgcctcacctccggcagcgatcacatagcccccggcgccgtggtggccctggtggacgagatcggctccgccgcctccgtgtccgacggccaccacctcaaggtctccgtcgacatgtccgtcaacttcgttgacctcgacgccgccgcgcccggggacgccctccgcatcaccgccagggcgctcggccacaagggcgcctactccggcacgcacgtcatcgtcgccaacgccgccacggggcaggtggtcgccgaggggaggcactcgctcttcggcaagatgaagatcaggagcaacatctgaattggagcatgtgtgcacacagtgctgtgtttgttgtcgtgtgctctgcttctcgactccttgtcannaatgtaatggcagcagctngcttcttcagtaaaaaatnaaaaaaaaannnnnnnannattatgtttnntcannntcatgatnnnnnnnancnnnnnnnnnnnnnnnncannnnnannnnnnnngat

SEQ ID NO: 123 - DNA, 446 bp, Zea mays
  unsure (1)..(446): n = a, t, c, or g
aaggtattttcagtcaagtggtggaaccaaaaggtagagaacatgaagtgggttttatttattttgatgagctcctgaaaagaggattgatccagcctgtagatacagactncaatgatcaagtgttgtcatgtatagttcatcaagtngtactggaatttattacaaagaaatcaatgcaggaaaacttcatcactgtcgtggattatcgtgaaacagggacagtgcttgatgataataaggttcatcgactgtctgccaggtttgaaggtgccaaaagtgcacagataccgcggagcttcagagtacgccaagttcggtcctttatgttttctggattcattaaatctttaccttcccttcncaagtattgccttgtccgagttctgnttcttcatgtttggagtgatgatcanggcaagactgtggttctngacctttctcca

SEQ ID NO: 124 - DNA, 679 bp, Zea mays
  unsure (1)..(679): n = a, t, c, or g
ggcggccaagcacaaggccgtggtggtgctgggcgccacgggcaccggcaagtcccgcctggcgatcgacctcgcgctgcgcttcggcggcgaggtcatcaactcggacaagatccaggcgcacgccggcctggacgtggccaccaacaaggtgggcctcgccgagcgcgggcgggtgccgcaccacctgctgggcgtggtgcacccggacgccgagttcacggcggccgacttccgccgcgaggcctnnnnnnnnnnnnnnnnnnnnnnnnnnnnnnnnnnnntgcccgtcatcgccggcggctccaactcgtacgtggaggagctcgtcgagggggaccggcgcgcgttccgggaccgctacgagtgctgcttcctctgggtggacgcgcagctccccgtgctgcacggcttcgtcgcccgccgcgtcgacgacatgtgccggcgcggcctcgtcgacgngntggnnnnnncgttcgacccccgnngcaccgactactccaggggcatctggcgcgccatcggcgtgccggagctcgangcctaccnncgggcgcgnnnnnnnnnnnncnnnnaccnnnacgnnnagatgcnnnnnnnnnnnnnnnnnnngnnnnnnnnnnnnnnnnnnnnnnnnnnnnnnnnnnnnnnnnnnnnnnnnnnnnnnnnnnnnnnnnna

SEQ ID NO: 125 - DNA, 21 bp, Artificial Sequence
  Other information Description of artificial sequence synthetic primer
ccaaccacgtgaagaccttca

SEQ ID NO: 126 - DNA, 24 bp, Artificial Sequence
  Other information Description of artificial sequence synthetic primer
ttgcgagtgagataaatctggaaa

SEQ ID NO: 127 - DNA, 21 bp, Artificial Sequence
  Other information Description of artificial sequence synthetic primer
catggcagcagacagtcaaca

SEQ ID NO: 128 - DNA, 20 bp, Artificial Sequence
  Other information Description of artificial sequence synthetic primer
cctccttggccaatgaacaa

SEQ ID NO: 129 - DNA, 26 bp, Artificial Sequence
  Other information Description of artificial sequence synthetic primer
gctgaaggtctctcaaatgtgtactg

SEQ ID NO: 130 - DNA, 26 bp, Artificial Sequence
  Other information Description of artificial sequence synthetic primer
caattgtacaccatatgctctagcaa

SEQ ID NO: 131 - DNA, 29 bp, Artificial Sequence
  Other information Description of artificial sequence synthetic primer
tgcacatctctactacttattgagacgat

SEQ ID NO: 132 - DNA, 18 bp, Artificial Sequence
  Other information Description of artificial sequence synthetic primer
gccgggaacgaacaacac

SEQ ID NO: 133 - DNA, 23 bp, Artificial Sequence
  Other information Description of artificial sequence synthetic primer
cgcttggtctggactctagaaaa

SEQ ID NO: 134 - DNA, 27 bp, Artificial Sequence
  Other information Description of artificial sequence synthetic primer
acagtcagtccctacagagtgtttagc

SEQ ID NO: 135 - DNA, 22 bp, Artificial Sequence
  Other information Description of artificial sequence synthetic primer
aggacgacgtggatagggatag

SEQ ID NO: 136 - DNA, 15 bp, Artificial Sequence
  Other information Description of artificial sequence synthetic primer
cgtcgggccgcatct

SEQ ID NO: 137 - DNA, 17 bp, Artificial Sequence
  Other information Description of artificial sequence synthetic primer
ggcacgcacgtcatcgt

SEQ ID NO: 138 - DNA, 21 bp, Artificial Sequence
  Other information Description of artificial sequence synthetic primer
cctgatcttcatcttgccgaa

SEQ ID NO: 139 - DNA, 21 bp, Artificial Sequence
  Other information Description of artificial sequence synthetic primer
gtacgccaagttcggtccttt

SEQ ID NO: 140 - DNA, 23 bp, Artificial Sequence
  Other information Description of artificial sequence synthetic primer
cagaactcggacaaggcaatact

SEQ ID NO: 141 - DNA, 18 bp, Artificial Sequence
  Other information Description of artificial sequence synthetic primer
cgtggaggagctcgtcga

SEQ ID NO: 142 - DNA, 19 bp, Artificial Sequence
  Other information Description of artificial sequence synthetic primer
cgtccacccagaggaagca

SEQ ID NO: 143 - DNA, 18 bp, Artificial Sequence
  Other information Description of artificial sequence synthetic probe
ccatacaaaactgtttct

SEQ ID NO: 144 - DNA, 17 bp, Artificial Sequence
  Other information Description of artificial sequence synthetic probe
ccatacaaaacagtttc

SEQ ID NO: 145 - DNA, 14 bp, Artificial Sequence
  Other information Description of artificial sequence synthetic probe
caacgttcgccgct

SEQ ID NO: 146 - DNA, 13 bp, Artificial Sequence
  Other information Description of artificial sequence synthetic probe
caacgctcgccgc

SEQ ID NO: 147 - DNA, 15 bp, Artificial Sequence
  Other information Description of artificial sequence synthetic probe
tcaggcaacattaaa

SEQ ID NO: 148 - DNA, 17 bp, Artificial Sequence
  Other information Description of artificial sequence synthetic probe
ttcaggtaacattaaac

SEQ ID NO: 149 - DNA, 14 bp, Artificial Sequence
  Other information Description of artificial sequence synthetic probe
atgcacatccatcc

SEQ ID NO: 150 - DNA, 14 bp, Artificial Sequence
  Other information Description of artificial sequence synthetic probe
atgcacaaccatcc

SEQ ID NO: 151 - DNA, 16 bp, Artificial Sequence
  Other information Description of artificial sequence synthetic probe
ttgtgaaggttgcctg

SEQ ID NO: 152 - DNA, 15 bp, Artificial Sequence
  Other information Description of artificial sequence synthetic probe
tttgtgaagattgcc

SEQ ID NO: 153 - DNA, 14 bp, Artificial Sequence
  Other information Description of artificial sequence synthetic probe
ccgaggacagtgtc

SEQ ID NO: 154 - DNA, 15 bp, Artificial Sequence
  Other information Description of artificial sequence synthetic probe
accgaggactgtgtc

SEQ ID NO: 155 - DNA, 14 bp, Artificial Sequence
  Other information Description of artificial sequence synthetic probe
ccaccagccccgtg

SEQ ID NO: 156 - DNA, 14 bp, Artificial Sequence
  Other information Description of artificial sequence synthetic probe
ccacctgccccgtg

SEQ ID NO: 157 - DNA, 16 bp, Artificial Sequence
  Other information Description of artificial sequence synthetic probe
tgttttctggattctt

SEQ ID NO: 158 - DNA, 18 bp, Artificial Sequence
  Other information Description of artificial sequence synthetic probe
tttctggattcattaaat

SEQ ID NO: 159 - DNA, 14 bp, Artificial Sequence
  Other information Description of artificial sequence synthetic probe
cgggaccggtacga

SEQ ID NO: 160 - DNA, 13 bp, Artificial Sequence
  Other information Description of artificial sequence synthetic probe
cgggaccgctacg